(19) United States
(12) Patent Application Publication
(10) Pub. No.: US 2004/0092470 A1
(43) Pub. Date: May 13, 2004

(54) Dry powder oligonucleotide formualtion, preparation and its uses

(76) Inventors: Sherry A. Leonard, Lawrenceville, NJ (US); Jonathan W. Nyce, Titusville, NJ (US)

Correspondence Address: HOWREY SIMON ARNOLD & WHITE, LLP, BOX 34, 301 RAVENSWOOD AVE., MENLO PARK, CA, 94025, US

(21) Appl. No.: 10/463,999

(22) Filed: Jun. 18, 2003

Related U.S. Application Data
(60) Provisional application No. 60/389,740, filed on Jun. 18, 2002.

(51) Int. Cl. A61K 48/00; C07H 21/04; A61L 9/04; A61K 9/14

(52) U.S. Cl. 514/44; 424/46; 536/23.2

(57) ABSTRACT

A formulation consisting essentially of an oligo(s) and bearing greater than about 90% particles about 0.1μ to about 1μ, or about 10μ to about 50μ in diameter. A dry powder formulation consisting essentially of an oligo of particle size about 0.1μ to about 100μ micron in diameter. Methods of preparation and therapeutic and diagnostic use are disclosed. Kits for diagnosis or treatment of numerous diseases and conditions by administration into the respiratory tract.

### BACKGROUND OF THE INVENTION

1. Field of the Invention

This invention relates to a dry powder oligonucleotide (oligo) formulation, to methods of preparing and for delivering the formulation. The formulation is suitable for treating a variety of ailments, including all types of diseases, including respiratory tract and lung diseases and conditions, allergy(ies), cancer and inflammation, among others.

2. Description of the Background

Inhalation therapy involves the administration of a drug in an aerosol form to the respiratory tract to the deep lung regions (alveoli), which account of 95% of lung epithelia, and provides significantly enhanced transport of the drug through the epithelial membrane.

Two general types of aerosol formulations have been employed for delivering small molecules, liquid aerosols and solid particulate aerosols. Liquid aerosols are generated by nebulization of solutions of a drug. Solid particulate aerosols are either in the form of a powder suspended or simply as a powder that is administered from a dry powder inhaler in a propellant that is administered from a metered dose inhaler. For some small molecules, solid particle aerosols intended for pulmonary administration are typically made by lyophilizing or freeze-drying a drug from solution and then milling or grinding the lyophilized drug to the desired particle size distribution.

Another way of formulating aerosol powders of therapeutic compounds is spray drying. Spray drying is a dehydration process that utilizes a hot gas stream, usually air, to evaporate dispersed droplets created by atomization of a continuous liquid feed of a compound. By this method, a product may be dried within a few seconds into fine particles, but in the case of biological macromolecules, it may lead to thermal denaturation and structural alterations attributed to loss of hydration water molecules required for preservation of secondary structure, physical forces such as shear, and changes in pH. To avoid these undesirable changes excipients are employed that can act as water-replacing agents, cryopreservatives, buffers, etc., for these macromolecules.

Spray drying has been applied to form microspheres of nucleic acids. In one method large biological polymers such as nucleic acids are dissolved in a solvent, sprayed into a freezing liquid, and the solvents are then extracted to form hardened microspheres. Although the polymers may contain active agents, the formulation is more suitable for delivering proteins, peptides, and other macromolecules to the lungs or nasal epithelium as an elixir or aerosol spray of particle size 10 to 80 micron. Liquid aerosol formulations have been employed for inhalation delivery of anti-sense oligonucleotides by one of the present inventors and others. Their administration, however, is difficult to handle and the liquid formulations must be freshly prepared because they have been found to exhibit a decreased activity when left for longer than a few hours.

A dry powder formulation of DNA molecules suitable for gene therapy has been disclosed, which has ontoward physical characteristics due to the large size of the DNA required. To be useful, this formulation requires the presence of cryoprotectant agents to facilitate lyophylization and powdering to a particle size no greater than 1 micron.

Anti-sense oligos are being tested currently for the treatment of a variety of diseases and have received theoretical consideration and experimental validation as pharmacological agents for treatment of human diseases. The administration of anti-sense oligo therapy via the respiratory tract has showed to have significant advantages for increasing target specificity and decreasing systemic side effects when compared with other routes of administration. Anti-sense oligo therapy is best administered to the treatment of respiratory diseases by delivering either DNA or RNA oligos via the respiratory system. Non-aqueous solvents have been used as an alternative to aqueous solvents, but they have proven to have drawbacks, such as carcinogenic and ozone depleting activities. Up to this time, however, no reports of effective non-aqueous dry powder formulations of therapeutic oligos have been reported.

Accordingly, there is a need for a dry powder formulation effective for delivering therapeutic oligos that shows high dispersibility and good respirability properties. The dry powder formulation of this invention makes it possible to efficiently deliver single and double stranded RNA and DNA oligonucleotides, including anti-sense oligos, to target organs and/or tissues via the respiratory tract.

### SUMMARY OF THE INVENTION

This invention relates to a dry powder formulation that comprises an oligonucleotide (oligo) and a pharmaceutically or veterinarily acceptable carrier or diluent, wherein greater than 80% of the particles of the agent are about 0.1 μm to about 100 μm in diameter. The oligo may be formulated by itself in the absence of carriers and cryoprotectants. The present formulation comprises an oligo(s) that may be simple or double stranded RNA or DNA or may be a chimeric nucleic acid. In addition, the oligo may be an anti-sense molecule that hybridizes to a nucleic acid target such as genes, the genes' initiation codons, genomic flanking regions, intron-exon borders, their 5′-end, or 3′-end, regions within 2 to 10 nucleotides of the 5′-end and the 3′-end, the sections extending over coding and non-coding regions, and coding and non-coding regions of RNAs corresponding to the target genes. The anti-sense oligo may be a STA (single target antisense) or a MTA (multi target antisense) oligo.

This invention also relates to methods for preparation of the dry powder formulation of the invention, by obtaining a dry pharmaceutical nucleic acid oligonucleotide (oligo) about 5- about 200 mononucleotides long, altering the particle size of the nucleic acid to form a dry formulation comprising greater than about 80% oligo particles of about 0.01 to about 1000μ in diameter, and from these selecting greater than 90% nucleic acid particles of about 0.1 to about 100μ in diameter.

The dry powder formulation of this invention may be delivered via the respiratory system, and depending on its particle size will be highly absorbed through the nasal mucose, or will penetrate to the lung(s). The present formulation may be administered alone or together with other ingredients and/or therapeutic agent(s), or co-jointly with the latter. The present formulation may be useful for preventing and/or treating inflammation, allergies, asthma, impeded airways or respiration, respiratory distress syndrome (RDS), Acute Respiratory Distress Syndrome (ARDS), side effects associated with the administration of adenosine and other therapeutic and diagnostic agents, cystic fibrosis (CF), pulmonary hypertension, pulmonary vasoconstriction, emphysema, chronic obstructive pulmonary disease (COPD), allergic rhinitis (AR), and cancers including leukemias, lymphomas and carcinomas of the lung and metastatic cancers.

The agent and formulation of the invention are provided in a kit suitable for use in animal and human experimentation, and in the diagnosis or treatment of a variety of diseases and conditions. The kit may comprise, in separate containers, a delivery device, the dry powder formulation of the invention, and instructions for loading the formulation into the device and for its use. Optionally other therapeutic or diagnostic agent(s), anti-oxidants, fillers, volatile oils, dispersants, anti-oxidants, flavoring agents, propellants, preservatives, solvents, buffering agents, RNA inactivating agents, cell internalized or up-taken agents, carriers or coloring agents may also be included along with instructions for their addition to the formulation and/or device prior to use. Suitable delivery devices are dry powder inhalers (DPI) and metered dose inhaler (MDI), the latter being a pressurized inhaler for delivery of dry powder formulation for delivering of particle sizes 0.1μ to 100μ. The kit may further comprise, in separate containers, propellant(s), and pressurized means for delivery adapted for delivering a dry powder formulation, and instructions for loading into the DPI device the dry powder formulation

### BRIEF DESCRIPTION OF THE DRAWINGS

FIG. 1 is a photograph showing particles obtained by spray drying.

### DETAILED DESCRIPTION OF THE PREFERRED EMBODIMENTS

This invention arose from a desire by the inventors to improve on prior art formulations employed for delivering oligonucleotides (oligos) via the respiratory system. Up to this time, oligos have been administered via the respiratory tract as aqueous aerosol formulations. Such formulations deliver generally no more than 10% of the content of oligo in the formulation to the respiratory tract tissues, the rest being wasted when the patient exhales either into the environment or as backflow into the delivery device. The inventors desired to improve the proportion of oligo delivered and absorbed by the patent. In order to attain this objective, the inventors thought of employing a dry powder formulation of limited particle sizes to improve the stability, and delivery of the formulation and the absorption of the oligo. The inventors are providing in this patent, thus, a dry powder formulation of an oligo(s) that shows good stability, and dispersibility as well as excellent respirable properties. The inventors found, surprisingly, that stable dry powder formulations of oligonucleotides (oligos) such as respirable antisense oligos (RASONs) are suitably prepared without the use of excipients or cryoprotectants. As is known in the art, drug formulations that will be frozen or lyophilized require the use of excipients such as carbohydrates, polypeptides, etc., for increasing stability during the freezing process, and to provide shelf stability. Surprisingly, the inventors found that their methods to produce highly stable and active dry powders for administration though the respiratory tract in the absence of such excipients.

The inventors have found that the success of a dry powder product, for example, for inhalation purposes is based on the ease of powder dispersibility, which is mainly determined by the efficiency of inhalation devices and by the physical properties of the powder being delivered. They considered that fact that many physical characteristics that affect the dispersibility of a powder, including the nature of the material, its particle size and distribution, its particle shape and morphology, and its moisture content. All these properties affect the inter-particle or cohesion forces and the particle-surface or adhesion forces. An increase in inter-particle cohesion tends to reduce powder segregation, and generally results in physically larger particles that are difficult to inhale into the deep lung. Accordingly, an increase in particle surface adhesion decreases powder flowability and increases powder retention of all contact surfaces. Because inertial deposition is often a dominant deposition mechanism, even when particles are physically small enough, e.g. less than 5μ, they are likely to deposit on the walls of the respiratory tract on their way down to the lung alveoli. The inventors concluded that sufficient inert particles would escape from the streamlines of air flow, and deposit on the airways. This invention is directed to the production of a formulation comprising particles that have low inertia while retaining their ability to reach the deep lung.

Glossary

As used herein, the term “adenosine-free”, as used herein, means that no adenosine residue is contained in an oligonucleotide, that is an adenosine-free oligonucleotide is devoid of adenosine. If any adenosine residue is present, it may be substituted or replaced with a mononucleotide other than A or an analogue or universal base to give a desA oligonucleotide. The term “agent”, as used herein, means a chemical compound, a mixture of chemical compounds, a synthesized compound, a therapeutic compound, an organic compound, an inorganic compound, a nucleic acid, a protein, a biological molecule, a macromolecule, lipid, oil, fillers, solution, a cell or a tissue. Agents may be added to prepare a formulation comprising an inhibitor or an oligonucleotide (oligo) and used in a composition, formulation or a kit for pharmaceutical or veterinary use. The term “airway”, as used herein, means part of or the whole respiratory system of a subject which is exposed to air. The airway includes the throat, trachea, nasal panage, sinuses, pharynx, windpipes, the respiratory tract, lungs, and the lung linings. The airway also includes the trachea, bronchi, bronchioles, terminal bronchioles, respiratory bronchioles, alveolar ducts, and alveolar sacs. The term “airway inflammation”, as used herein, means a disease or condition associated with inflammation of the airways of a subject. Airway inflammation may be caused or accompanied by allergy(ies), asthma, impeded airways or respiration, cystic fibrosis (CF), Chronic Obstructive Pulmonary Diseases (COPD), allergic rhinitis (AR), Acute Respiratory Distress Syndrome (ARDS), respiratory distress syndrome (RDS), pulmonary hypertension, lung inflammation, bronchitis, airway obstruction, bronchoconstriction and/or infections such as viral and bacterial infections, among other respiratory tract problems. The term “anti-sense oligonucleotide (oligo)”, as used herein, means an oligonucleotide that is applied to the reduction or inhibition of gene expression by inhibition of a target nucleic acid. The target nucleic acid is preferably messenger RNA (mRNA) or a gene(s). The oligonucleotide (oligo) generally means a sequence of about 5 to about 200 synthetic or naturally derived mononucleotide that (1) hybridizes to a segment of an mRNA encoding a target protein under appropriate hybridization conditions, and that (2) upon hybridization causes a reduction in gene expression of the target protein. There may be DNA or RNA, and single or double stranded. See, for example, Milligan, J. F. et al., J. Med. Chem. 36(14): 1923-1937 (1993), the relevant portion of which is hereby incorporated in its entirety by reference. The term “carrier”, as used herein, means a biologically acceptable carrier in the form of gaseous, liquid, solid carriers, and/or mixtures thereof, that are suitable for administration by the intended routes. The carrier is preferably a pharmaceutically or veterinarily acceptable carrier. The composition may optionally comprise other agents such as other therapeutic compounds known in the art for the treatment of the condition or disease, antioxidants, flavoring agents, coloring agents, fillers, volatile oils, buffering agents, dispersants, surfactants, RNA inactivating agents, propellants and preservatives, as well as other agents known to be utilized in therapeutic compositions. The term “cell-internalized agent”, as used herein, means an agent that enhances or facilitates the internalization of a desired compound or composition into a cell. Examples of cell-internalized agents are transferrin, asialoglycoprotein, streptavidin, or sperimine, among others. The term “chimeric” oligonucleotides or “chimeras”, as used herein, means oligonucleotides (oligos) which contain two or more chemically distinct regions, at least one of which is made up of nucleotides.

The term “complementary”, as used herein, means the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and a DNA or RNA molecule are said to be complementary to each other when a sufficient number of corresponding positions in each molecule are occupied by nucleotides that hydrogen bond with each other. The term “composition”, as used herein, means a mixture containing a dry powder formulation comprising an oligo used in this invention, and optionally a carrier and/or other agents. The composition is preferably a pharmaceutical or veterinary composition. The terms “des-adenosine (desA)” and “des-thymidine (desT)”, as used herein, mean oligonucleotides (oligos) substantially lacking either adenosine (A), uridine (U), or thymidine (T), respectively. In some instances, the desT, desU, or desA sequences are naturally occurring, and in others they may result from substitution to eliminate the presence of an undesirable adenosine (A), uridine (U), or thymidine (T), nucleotide to avoid its undesirable activity, e.g., for A at the adenosine receptor(s). In the present context, the substitution is generally accomplished by substitution of A, or other mononucleotides, e.g., guanidine (G), cytosine (C), U or T, with a “universal base”, as is known in the art. The term “down-regulation” as used herein, means a decrease in production, secretion, expression or availability (and thus a decrease in concentration) of a gene product, including targeted protein or nucleic acids. The term “an effective amount” as used herein, means an amount that provides a therapeutic or prophylactic benefit. The term “fixed” as used herein, means that a non-homologous nucleotide may be replaced with a universal base that may base-pair with similar or equal affinity with two or more of the four nucleotide present in natural DNA: A (adenine), G (guanine), C (cytosine), and T (thymidine). This step generates a further novel sequence, different from the one found in nature, that permits the oligonucleotide (oligo) to bind, preferably equally well, with the primary target, a secondary target, a tertiary target, etc. The term “fragment”, as used herein, means a single- or double-stranded nucleic acid, be it of DNA or RNA, having a desired sequence. The fragment has at least four contiguous mononucleotides having a sequence derived from a desired source. The term “homology”, as used herein, means the identity of residues in nucleic acid or amino acid sequences. A 100% identity of two or more sequences, indicates that those sequences have identical residues. The term “homologous”, as used herein, means that one single-stranded nucleic acid sequence may hybridize to a complementary single-stranded nucleic acid sequence. The degree of hybridization may depend on a number of factors including the degree of identity between the sequences and the hybridization conditions such as temperature and salt concentration, as discussed later. The region of identity is preferably greater than about 5 base pairs (bp), more preferably greater than about 7 bp, and still more preferably greater than about 10 bp. “Homologous”, thus, means the level of sequence identity, preferably, about 60% or more, preferably about 70% or more, preferably about 80% or more, more preferably about 90% or more, or most preferably any one of about 95%, about 96%, about 97%, about 98% or about 99%. Residues that are not identical are mismatches. The term “hybridize”, as used herein, means that a nucleic acid including an oligonucleotide (oligo) binds to its complementary chain of a nucleic acid and maintains binding under appropriate conditions. Hydrogen bonding, either Hoogsteen hydrogen bonding or Watson-Crick hydrogen bonding, is formed between complementary nucleoside or nucleotide bases. Adenine and thymidine for example, are complementary nucleotide bases, and cytosine and guanine are complementary nucleotide bases that pair through the formation of hydrogen bonding. If a complementary chain is not homologous, a nucleic acid may not bind to and/or form a bonding. The term “methylated cytosine” (^{m}C), as used herein, means a methylated cytosine base that is substituted for cytosine (C) to create at least one methylated CpG (^{m}CpG) dinucleotide present in an oligonucleotide (oligo). The term “oligonucleotide (oligo)”, as used herein, means an oligomer or polymer of ribonucleic acid (RNA) or deoxyribonucleic acid (DNA), or mimetics thereof. This term includes oligonucleotides composed of naturally-occurring nucleobases, sugars and covalent intersugar (backbone) linkages as well as oligonucleotides having non-naturally-occurring portions which function similarly, that may be single- or double-stranded. Such modified or substituted oligonucleotides are often preferred over native forms because of desirable properties such as, for example, enhanced cellular uptake, enhanced binding to target and increased stability in the presence of nucleases. Preferably, an oligonucleotide is 1-200 mononucleotides or analogues in length, preferably about 4 to 70, 7 to 70, 7 to 60, 10 to 50, 20 to 40, 20 to 30, 21, 22, 23, 24, 25, 26, 27, 28, or 29, in length. The oligonucleotide may be preferably an anti-sense oligonucleotide. The term “multi-targeted anti-sense (MTA) oligonucleotide (oligo)”, as used herein, means an oligonucleotide that hybridizes to at least two different nucleic acids and is capable of attenuating the expression of more than one target gene or mRNA, or to enhance or attenuate the activity of one or more pathways. The term “naturally-occurring”, as used herein, means the fact that an object may be found in nature. For example, a nucleic acid or a nucleic acid sequence that is present in an organism (including viruses) that may be isolated from a source in nature and that has not been intentionally modified by man in the laboratory is said to be naturally-occurring. The term naturally-occurring generally refers to an object as present in a non-pathological (undiseased) individual, such as would be typical for the species. The term “non-fully desA sequence”, as used herein, means a sequence that may have a content of adenosine of less than about 15%, more preferably less than about 10%, and still more preferably less than 5%, and some even less than 2% adenosine.

The term “operatively (operably) linked”, as used herein, means that a nucleic acid is placed into a functional relationship with another nucleic acid sequence including a presequence, secretory leader sequence, promoter, enhancer, ribosome binding site, expression control sequence, or reporter gene, etc. Generally, “operatively linked” means that the DNA sequences being linked are contiguous, for some sequences and, not for other sequences. Linking is accomplished by ligation at convenient restriction sites. If such sites do not exist, the synthetic oligonucleotide adaptors or linkers are used in accordance with conventional practice. The terms “preventing” or “prevention”, as used herein, mean a prophylactic treatment made before a subject obtains a disease or ailing condition such that it can have a subject avoid having a disease or condition related thereto. The term “reducing”, as used herein, means decreasing or preventing the translation or expression of a gene by an oligonucleotide that binds specifically with a target mRNA. The term “respiratory diseases”, as used herein, means diseases or conditions related to the respiratory system. Examples include, but not limited to, airway inflammation, allergy(ies), asthma, impeded respiration, cystic fibrosis (CF), Chronic Obstructive Pulmonary Diseases (COPD), allergic rhinitis (AR), Acute Respiratory Distress Syndrome (ARDS), pulmonary hypertension, lung inflammation, bronchitis, airway obstruction, infections, such as viral bacterial and the like and bronchoconstriction. The terms “a segment”, as used herein, means at least four contiguous nucleotides having a sequence derived from any part of mRNA. The term “sequence identity”, as used herein, means that two polynucleotide sequences are homologous or identical, i.e., on a nucleotide-by-nucleotide basis over the window of comparison. The term “percentage of sequence identity” or “homology” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison, i.e. the window size, and multiplying the result by 100 to yield the percentage of sequence identity. The term “substantial identity”, as used herein means a characteristic of a polynucleotide sequence, wherein the polynucleotide comprises a sequence that has at least 80% sequence identity, preferably at least 85% identity and often 90 to 95% sequence identity, more usually at least 99% sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20% or less of the reference sequence over the window of comparison.

The term “a spacer”, as used herein, means a molecule or a group of molecules that connects two molecules, such as a nucleotide and a random nucleotide, and serves to place the two molecules in a preferred configuration. The term “a target”, as used herein, means a nucleic acid, a gene cDNA clones, mRNA, or a gene product or protein to which an inhibitor used in this invention acts on. For example, an oligonucleotide targeting to a specific nucleic acid hybridizes to its target nucleic acid and suppresses the expression of a target gene, thereby production of the target protein is inhibited. The terms “treat” or “treating”, as used herein, mean a treatment which decreases the likelihood that the subject administered such treatment will manifest symptoms of disease or other conditions.

The term “universal base”, as used herein, means a substitute base used for a mononucleotide, whether A, T, G or C, in its position in a nucleic acid which forms a hydrogen bond and binds to the complementary base but lacks the ability to activate replaced mononucleotide's receptors and otherwise exercise its constricting effect in the lungs. The term “up-regulation”, as used herein, means an increase in production, secretion, expression, function or availability, and thus an increase in concentration, of the gene product, e.g. targeted protein or nucleic acids. The term “an up-taken agent”, as used herein, means an agent which helps a cell take up a substance into a cell. It is used to take an exogenous substance into a cell to passively give a different genotype and/or phenotype. Examples of up-taken agents are transferrin, asialoglycoprotein, streptavidin, or sperimine, although others are also suitable.

This invention, thus, provides a method for preparing a dry powder formulation of an oligo(s), comprising obtaining a dry pharmaceutical agent comprising an oligo of 1 to about 200 mononucleotides, altering the particle size of the agent to form a dry formulation of particle size about 0.1 μm to about 1000 μm in diameter, and selecting particles of the formulation comprising greater than about 90% oligo particles of about 0.1 μm to about 100 μm in diameter.

Known methods are utilized in obtaining a dry pharmaceutical agent comprising an oligo. The oligos in the dry pharmaceutical agent used in this invention are preferably antisense oligo for diagnostic and/or therapeutic purposes having 1 to 200 mononucleotides length, preferably 10-100, more preferably 10-40, most preferably 20-30, and 20, 21, 22, 23, 24, 25, 26, 27, 28, 29, or 30 mononucleotides are preferably exemplified. The oligo is substantially prepared as a dry pharmaceutical agent. The dry pharmaceutical agent may be obtained in solid form and the solid agent comprises powder. However, the pharmaceutical agent may be placed in a solution, suspension or emulsion, and can comprise one or more formulation ingredients.

A mononucleotide comprises a nucleoside or base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligos, the phosphate groups covalently link adjacent nucleosides to one another to form a linear polymeric compound. In turn the respective ends of this linear polymeric structure can be further joined to form a circular structure, however, open linear structures are generally preferred. Within the oligo structure, the phosphate groups are commonly referred to as forming the internucleoside backbone of the oligo. The normal linkage or backbone of RNA and DNA is a 3′ to 5′ phosphodiester linkage. Specific examples of preferred antisense compounds useful in this invention include oligos containing modified backbones or non-natural internucleoside linkages. As defined in this specification, oligo(s) having modified backbones include those that retain a phosphorus atom in the backbone and those that do not have a phosphorus atom in the backbone. For the purposes of this specification, and as sometimes referenced in the art, modified oligo(s) that do not have a phosphorus atom in their internucleoside backbone can also be considered to be oligo(s). Preferred modified oligo backbones include, for example, phosphorothioate, chiral phosphorothioate, phosphorodithioate, phosphotriester, aminoalkylphosphotriester, methyl phosphonate, 3′-alkylene phosphonate, chiral phosphonate, phosphinate, phosphoramidate, 3′-amino phosphoramidate, aminoalkylphosphoramidate, thionophosphoramidate, thionoalkylphosphonate, thionoalkylphosphotriester, boranophosphate, morpholino, siloxane, sulfide, sulfoxide, sulfone, formacetyl, thioformacetyl, methylene formacetyl, thioformacetyl, alkene, sulfamate, methyleneimino, methylenehydrazino, sulfonate, sulfonamide, amide, thioether, carbonate, carbamate, sulfate, sulfite, hydroxylamine, methylene(methylimino), methyleneoxy (methylimino), 2′-O-methyl, or phosphoramidate having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. Representative patents describing the preparation of the above phosphorus-containing linkages include, but are not limited to U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the relevant patent of all of which is incorporated herein by reference.

Preferred modified oligo backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Other preferred modified oligo backbones have thioether, carbonate, carbamate, sulfate, sulfite, hydroxylamine, methylene(methylimino), methyleneoxy (methylimino), 2′-O-methyl, and phosphoramidate backbones. Representative patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, the relevant patent of all of which is incorporated herein by reference. In other preferred oligo mimetics, both the sugar and the internucleoside linkage, i.e. the backbone, of the nucleotide units are replaced with novel groups. The base units are maintained for hybridization with an appropriate nucleic acid target compound. One such oligomeric compound, an oligo mimetic that has been shown to have excellent hybridization properties, is referred to as a peptide nucleic acid (PNA). In PNA compounds, the sugar-backbone of an oligo is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; 5,719,262; Nielsen et al. See Science 254: 1497-1500 (1991), the relevant patent of all of which is incorporated herein by reference. Most preferred embodiments used in the invention are oligo(s) with phosphorothioate backbones and oligonucleosides with heteroatom backbones, and in particular, —CH₂NHOCH₂—, —CH₂N(CH₃)OCH₂— (known as a methylene (methylimino) or MMI backbone), —CH₂ON(CH₃)CH₂—, —CH₂ N(CH₃)N(CH₃)CH₂— and —ON(CH₃)CH₂CH₂—, wherein the native phosphodiester backbone is represented as —OPOCH₂—, of U.S. Pat. No. 5,489,677, and the amide backbones of U.S. Pat. No. 5,602,240. Also preferred are oligos having morpholino backbone structures described in U.S. Pat. No. 5,034,506. Modified oligo(s) may also contain one or more substituted sugar moieties. Preferred oligo(s) comprise one of the following at the 2′ position: OH; F; O-, S-, or N-alkyl, O-alkyl-O-alkyl, O-, S-, or N-alkenyl, or O-, S-, or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)₂ON(CH₃)₂, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligos comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OC₃, SOCH₃, SO₂CH₃, ONO₂, NO₂, N₃, NH₂, heterocycloalkyl, heterocycloalkaryl, aminoalkylamino, poly-alkylamino, substituted silyl, an RNA cleaving group, a reporter group, an intercalator, a group for improving the pharmacokinetic properties of an oligo, or a group for improving the pharmacodynamic properties of an oligo, and other substituents having similar properties. A preferred modification includes 2′-methoxyethoxy (2′—O—CH₂CH₂OCH₃, also known as 2′—O-(2-methoxyethyl) or 2′-MOE) (Martin et al., Helv. Chim. Acta 1995, 78, 486-504) i.e., an alkoxyalkoxy group. Further preferred modifications include 2′-dimethylaminooxyethoxy, i.e., a O(CH₂)₂ON(CH₃)₂ group, also known as 2′-DMAOE, and 2′-dimethylaminoethoxyethoxy (2′-DMAEOE) as described in examples hereinbelow. Other preferred modifications include 2′-methoxy (2—O—CH₃), 2′-aminopropoxy (2′—OCH₂CH₂CH₂NH₂) and 2′-fluoro (2′—F). Similar modifications may also be made at other positions on the oligo, particularly the 3′ position of the sugar on the 3′ terminal nucleotide or in 2′-5′ linked oligos and the 5′ position of 5′ terminal nucleotide. Oligos may also have sugar mimetics such as cyclobutyl moieties in place of the pentofuranosyl sugar. Also Locked Nucleic Acid (LNA) and morpholino may be applicable for sugar mimetics. Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, the relevant patent of all of which is incorporated herein by reference. Oligo(s) may also include nucleobases often referred to in the art simply as a “base”, modifications or substitutions. As used herein, “unmodified” or “natural” nucleobases include the purine bases adenine (A) and guanine (G), and the pyrimidine bases thyimine (T), cytosine (C) and uracil (U). Modified nucleobases include other synthetic and natural nucleobases such as 5-methylcytosine (^{mc}C or ^{m}C), 5-hydroxymethyl cytosine, xanthine and its derivatives (e.g., theophylline, caffeine, dyphylline, etophylline, acephylline piperazine, bamifylline, and enprofylline), hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, Concise Encyclopedia of polymer Science & Engineering, pp 858-859 (1990), Kroschwitz, J. I., Ed., John Wiley & Sons, Englisch et al., Angewandte Chemie, International Ed. 30: 613-722 (1991), Sanghvi, Y. S., Chapter 15, Antisense Research and Applications 1993, pp 289-302, Crooke, S. T. and Lebleu, B., Eds., CRC Press, the relevant patent of all of which is incorporated herein by reference. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by about 0.6 to about 1.2 degree. C. See, for example, Sanghvi, Y. S., Crooke, S. T. and Lebleu, B., Eds., Antisense Research and Applications, CRC Press, Boca Raton, pp 276-278 (1993) and are presently preferred base substitutions, even more particularly when combined with 2′-O-methoxyethyl sugar modifications, the relevant patent of all of which is incorporated herein by reference. Representative patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, the relevant patent of all of which is incorporated herein by reference. Another modification of the oligo(s) used in the invention involves chemically linking to the oligo one or more moieties or conjugates which enhance the activity, cellular distribution or cellular uptake of the oligo. Such moieties include but are not limited to lipid moieties such as a cholesterol moiety, Letsinger et al., P.N.A.S. (USA) 86: 6553-6556 (1989), cholic acid (Manoharan et al., Bioorg. Med. Chem. Lett. 4: 1053-1059 (1994), a thioethers, e.g., hexyl-S-tritylthiol (Manoharan et al., Ann. N.Y. Acad. Sci. 660: 306-309 (1992); Manoharan et al., Bioorg. Med. Chem. Letters 3: 2765-2770 (1993), a thiocholesterols, Oberhauser et al., Nucl. Acids Res. 20: 533-538 (1992), aliphatic chains, e.g. dodecandiol or undecyl residues (Saison-Behmoaras et al., EMBO J. 10: 1111-1118 (1991), Kabanov et al., FEBS Letters 259: 327-330 (1990); Svinarchuk et al., Biochimie 75: 49-54 (1993), phospholipids, e.g. di-hexadecyl-rac-glycerol or triethylammonium 1,2-di-O-hexadecyl-rac-glycero-3-H-phosphonate (Manoharan et al., Tetrahedron Letters 36: 3651-3654 (1995); Shea et al., Nucl. Acids Res. 1990, 18, 3777-3783), a polyamine or a polyethylene glycol chain (Manoharan et al., Nucleosides & Nucleotides 1995, 14, 969-973), or adamantane acetic acid (Manoharan et al., Tetrahedron Lett. 1995, 36, 3651-3654), a palmityl moiety (Mishra et al., B. B. A., 1264: 229-237 (1995), or octadecylamine or hexylamino-carbonyl-oxycholesterol moieties (Crooke et al., J. P. E. Therap., 277: 923-937 (1996), the relevant patent of all of which is incorporated herein by reference. Representative patents for preparation of such oligo conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, the relevant patent of all of which is incorporated herein by reference.

This invention also includes a use of oligo(s) which are chimeric or mixed oligos. These oligos typically contain at least one region wherein the oligo is modified so as to confer upon the oligo increased resistance to nuclease degradation, increased cellular uptake, and/or increased binding affinity for the target nucleic acid. An additional region of the oligo may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of antisense inhibition of gene expression. Cleavage of the RNA target can be routinely detected by gel electrophoresis and, if necessary, associated nucleic acid hybridization techniques known in the art. This RNAse H-mediated cleavage of the RNA target is distinct from the use of ribozymes to cleave nucleic acids which are also included in this invention. Examples of chimeric oligos include but are not limited to “gapmers,” in which three distinct regions are present, normally with a central region flanked by two regions that are chemically equivalent to each other but distinct from the gap. A preferred example of a gapmer is an oligo in which a central portion (the “gap”) of the oligo serves as a substrate for RNase H and is preferably composed of 2′-deoxynucleotides, while the flanking portions (the 5′ and 3′ “wings”) are modified to have greater affinity for the target RNA molecule but are unable to support nuclease activity (e.g., fluoro- or 2′-O-methoxyethyl-substituted). Chimeric oligos are not limited to those with modifications on the sugar, but may also include oligonucleosides or oligos with modified backbones, e.g., with regions of phosphorothioate and phosphodiester backbone linkages or with regions of MMI and phosphorothioate backbone linkages. Other chimeras include “wingmers,” also known in the art as “hemimers,” that is, oligos with two distinct regions. In a preferred example of a wingmer, the 5′ portion of the oligo serves as a substrate for RNase H and is preferably composed of 2′-deoxynucleotides, whereas the 3′ portion is modified in such a fashion so as to have greater affinity for the target RNA molecule but is unable to support nuclease activity (e.g., 2′-fluoro- or 2′-O-methoxyethyl-substituted), or vice-versa. In one embodiment, the oligos of this invention contain a 2′-O-methoxyethyl (2′—O—CH₂CH₂OCH₃) modification on the sugar moiety of at least one nucleotide. This modification has been shown to increase both affinity of the oligo for its target and nuclease resistance of the oligo. According to the invention, one, a plurality, or all of the nucleotide subunits of the oligos of the invention may bear a 2′-O-methoxyethyl (OCH₂CH OCH₂) modification. Oligos comprising a plurality of nucleotide subunits having a 2′-O-methoxyethyl modification can have such a modification on any of the nucleotide subunits within the oligo, and may be chimeric oligos. Aside from or in addition to 2′-O-methoxyethyl modifications, oligos containing other modifications which enhance antisense efficacy, potency or target affinity are also preferred. Chimeric oligos comprising one or more such modifications are presently preferred.

The oligos used in this invention may be conveniently and routinely made through the well-known technique of solid phase synthesis. Equipment for such synthesis is sold by several vendors including Applied Biosystems. Any other means for such synthesis may also be employed and the actual oligo synthesis is well within preview of the artisan. Similar techniques are known to prepare oligos such as the phosphorothioates and 2′-alkoxy or 2′-alkoxyalkoxy derivatives, including 2′-O-methoxyethyl oligos. See, for example, Martin, P., Helv. Chim. Acta 78: 486-504 (1995). Similar techniques and commercially available modified amidites and controlled-pore glass (CPG) products such as biotin, fluorescein, acridine or psoralen-modified amidites and/or CPG (Glen Research, Sterling, Va.) may also be employed to synthesize fluorescently labeled, biotinylated or other conjugated oligos, as is known in the art. Also included in this patent are bioequivalent compounds, including pharmaceutically acceptable salts and prodrugs, such as pharmaceutically acceptable salts, esters, or salts of such esters, or any other compounds that, upon administration to an animal including a human, provide directly or indirectly a biologically active metabolite or residue thereof. “Pharmaceutically acceptable salts” are physiologically and pharmaceutically acceptable salts of the nucleic acids of the invention, i.e. salts that possess biological activity and do not impart undesired toxicological effects thereto. See, for example, Berge et al., Pharmaceutical Salts, J. Pharm. Sci. 66: 1-19 (1977), the relevant patent of all of which is incorporated herein by reference. Examples of pharmaceutically or veterenarily acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. Others, however, are also within the four corner of this patent. The oligos of the invention may be prepared in a “prodrug” form. The term “prodrug” indicates a therapeutic agent that is prepared in an inactive form that is converted to an active form, i.e. a drug, within the body or cells by the action of endogenous enzymes or other chemicals and conditions. In particular, prodrug versions of the present oligos may be prepared as SATE ((S-acetyl-2-thioethyl) phosphate) derivatives. See, WO 93/24510 to Gosselin et al., the relevant patent of all of which is incorporated herein by reference.

The single target antisense (STA) and multi target antisense (MTA) oligos of this invention attenuate the expression of one or more target mRNA(s), or enhance or attenuate the activity of one or more pathways. By means of example, this method may be practiced by first identifying all possible antisense sequences of about 7, about 10, about 12, about 15, about 18, about 19 to about 31, about 32, about 35, about 40, about 45, about 50, about 60, about 70 or more mononucleotides in a target mRNA. This may be attained by searching for segments that are 7 or more nucleotides long within a target sequence that are low in, or lack, thymidine (T) or uridine (U), a nucleotide which is complementary to adenosine (A). Although T, U and A are used as an example, the narrative applies to the substitution of any and all mononucleotides. This search typically results in about 10 to about 30 such desT or desU segments, i.e. these naturally lacking thymidine or uridine, or segments with low T or U content, e.g. up to and including about 20%, about 15% T or U, from which oligos of varying lengths may be designed for a typical target mRNA of average length, i.e. about 1800 nucleotides long. The sense sequence for each strictly complementary desA anti-sense oligo sequence obtained for a specific target may be then deduced, and used to search for sequences of preferred secondary targets. Alternatively, one or more sequence databases, e.g., GENBANK, and the like, may be searched for alternative secondary sequences. Thus, the targeting may be undertaken in several manners, one being the selection of specific targets associated with one or more related diseases. Alternatively, a primary target may be selected first, and an oligo found, preferably, a desA oligo and, then, secondary, tertiary or more targets searched for if an MTA is desired. In a typical search, either the list of preferred secondary targets or of a database, multiple instances of homologous secondary targets of interest are identified. That is, this technology is directed to finding the instances where there are natural homologies between primary, secondary, and other target sequences, and utilizing the finding for designing antisense oligos for preventative and therapeutic treatment of specific diseases or conditions associated with the target macromolecules from which the MTAs are obtained.

In this invention, the design of an oligo(s) targeted to a mRNA(s) associated with an ailment(s), for example, lung airway pathology(ies), and their modification may be designed to reduce the occurrence of undesirable side effects caused by their release of adenosine upon breakdown, while preserving their activity and efficacy for their intended purpose. In this manner, the inventors target a specific gene to design one or more oligo(s) that selectively bind(s) to the corresponding mRNA(s), and then reduces, if necessary, their content of adenosine via substitution with a universal base(s) or adenosine analog(s) incapable of activating adenosine A₁, A_{2b} or A₃ receptors. Based on the prior experience in the field, the inventors reasoned that in addition to “down-regulating” a specific gene(s), they could increase the effect of the oligo(s) administered by either selecting a segment of RNA(s) that is(are) devoid, or has(have) a low content, of thymidine (T) or, alternatively, substitute one or more adenosine(s) present in the designed oligo(s) with an other nucleotide base(s), so called universal base(s), which bind(s) to thymidine but lack(s) the ability to activate the indicated adenosine receptors and otherwise exercise the constricting effect of adenosine in the lungs, etc. Given that adenosine (A) is a nucleotide base complementary to thymidine (T) and uridine (U), when a T appears in the gene or RNA sequence, the oligo will have an A at the same position. For consistency's sake, all RNAs and oligos are represented in this patent by a single strand in the 5′ to 3′ direction, when read from left to right, although their complementary sequence(s)and the double-stranded oligo(s) is(are) also encompassed within the four corners of the patent, where all mononucleotides and amino acids are represented utilizing the recommendations of the IUPAC-IUB Biochemical Nomenclature Commission, or by the known 3-letter code (for amino acids). The oligo(s) of the invention may be utilized to treat ailments, for example, ones associated with airway inflammation which may be accompanied by reduced airway function in a subject, whatever its cause. The oligo(s) of the invention may have a reduced A content to prevent its liberation upon in vivo degradation of the oligo(s). Examples of airway diseases that may be treated by the method of this invention include airway inflammation, allergy(ies), asthma, impeded respiration, cystic fibrosis (CF), Chronic Obstructive Pulmonary Diseases (COPD), allergic rhinitis (AR), Acute Respiratory Distress Syndrome (ARDS), and/or bronchoconstriction. In addition, when designing MTAs, other mononucleotides may be replaced, as described above, in order to attain suitable hybridization to two or more targets. As a suitable gene is selected as a target, its mRNA or DNA is searched for low thymidine or thymidine-free (desT) fragments. Only desT segments of the mRNA or DNA are selected which, in turn, will produce desA antisense as their complementary strand. When a number of RNA desT segments are found, the sequence of the antisense segments may be deduced. Typically, about 10 to about 30 and even larger numbers of desA antisense sequences may be obtained. These antisense sequences may include some or all desA oligo sequences corresponding to desT segments of the mRNA of the target. When this occurs, the oligos found are said to be 100% A-free. For each of the original desA oligo sequences corresponding to the target gene, typically about 10 to 30 sequences may be found within the target gene or RNA that have a low content of thymidine (RNA). In accordance with this invention, the selected fragment(s) may also contain a small number of uridine nucleotides within the secondary or tertiary or quaternary sequences (RNA). In some cases, a large adenosine content may suffice to render the oligo less active or even inactive against the target. Thus, the replacement of nucleotides may be done to decrease the A content of the antisense oligo and/or to increase hybridization to a plurality of targets. In this invention, these so called “non-fully desA” sequences may preferably have a content of adenosine of less than about 15%, more preferably less than about 10%, and still more preferably less than 5%, and some even less than 2% adenosine. In some instances a higher content of adenosine is acceptable and the oligos are still active, particularly where the adenosine nucleotide may be “fixed” or replaced with a “universal” base that may base-pair with similar or equal affinity to two or more of the four nucleotide present in natural DNA:A, G, C, and T. A universal base is defined in this patent as any compound comprising an analogue, having the capacity to hybridize to the complementary base(s) at the target site, preferably having substantially reduced, or substantially lacking, ability to bind to the eliminated mononucleotide receptors. Alternatively, analogs that do not activate adenosine receptors, such as the adenosine A₁, A_{2 b} and/or A₃ receptors, most preferably A₁ receptors, may be used. One example of a universal base is 1-(2′-deoxy-β-D-ribofuranosyl)-5-nitroindole, and an artisan will know how to select others. This “fixing” step generates a further novel sequence, different from the one found in nature, that permits the oligo to bind, preferably equally well, with the target RNA. An example of a universal base is 1-(2′-deoxy-β-D-ribofuranosyl)-5-nitroindole. Other examples of universal bases are 1-(2′-deoxy-β-D-ribofuranosyl)-3-nitropyrrole, 7-(2′-deoxy-′-D-ribofuranosyl)inosine, 7-(2′-deoxy-β-D-ribofuranosyl)nebularine, 6H,8H-3,4-dihydropyrimido[4,5-c]oxazine-7-one-2′-deoxyribose and 2-amino-6-methoxyaminopurine (Glen Research, Sterling, Va.). In addition to the above, universal bases which may be substituted for any other base although with somewhat reduced hybridization potential, include 1-(2′-deoxy-β-D-ribofuranosyl)-3-nitropyrrole, 1-(2′-deoxy-β-D-ribofuranosyl)-5-nitroindole, 7-(2′-deoxy-β-D-ribofuranosyl)inosine, 7-(2′-deoxy-β-D-ribofuranosyl)nebularine, 7-(2′-deoxy-β-D-ribofuranosyl)isoguanosine, 7-(2′-deoxy-β-D-ribofuranosyl)-4-methylindole, 7-(2′-deoxy-β-D-ribofuranosyl)-6-phenylinosine, 7-(2′-deoxy-β-D-ribofuranosyl)-2,6-diamino-purine (TriLink BioTechnologies, San Diego, Calif.). More specific mismatch repairs may be made using “P” nucleotide, 6H,8H-3,4-dihydropyrimido[4,5-c][1,2]oxazin-7-one-2′deoxyribose, which base pairs with either guanine (G) or adenine (A) and “K” nucleotide, 2-amino-6-methoxyaminopurine, which base pairs with either cytidine (C) or thymidine (T), among others.

Others known in the art are also suitable. See, for example, Loakes, D. and Brown, D. M., Nucl. Acids Res. 22:4039-4043 (1994); Ohtsuka, E. et al., J. Biol. Chem. 260(5):2605-2608 (1985); Lin, P.K.T. and Brown, D. M., Nucleic Acids Res. 20(19):5149-5152 (1992; Nichols, R. et al., Nature 369(6480): 492-493 (1994); Rahmon, M. S. and Humayun, N. Z., Mutation Research 377 (2): 263-8 (1997); Amosova, O., et al., Nucleic Acids Res. 25 (10): 1930-1934 (1997); Loakes D. & Brown, D. M., Nucleic Acids Res. 22 (20): 4039-4043 (1994), the entire sections relating to universal bases and their preparation and use in nucleic acid binding being incorporated herein by reference. When non-fully desT or desU sequences are found in the naturally occurring target or when a mononucleotide is to be replaced (as for MTAs), they typically are selected so that about 1 to 3 universal base substitutions will suffice to obtain a 100% “desA” oligo. Thus, this method employs either oligos that are anti-sense to different targets that are low in, or devoid of, A content, or oligos where one or more adenosine nucleotides, e. g. about 1 to 3, may be “fixed” by replacement with a “universal” base, as in MTA design. Universal bases are known in the art and need not be listed herein. An artisan will know which bases may act as universal bases, and replace them for A. The oligo used in this invention is concerned primarily with the utilization for vertebrates, and within this group, of mammals, including human and non-human simians, wild and domesticated animals, marine and land animals, household pets, and zoo animals, for example, felines, canines, equines, pachyderms, cetaceans, and still more preferably to human subjects. One particularly suitable application of this technology is for veterinary purposes, and includes all types of small and large animals in the care of a veterinarian, including wild animals, marine animals, household animals, zoo animals, and the like. Targeted genes and proteins are preferably mammalian, and the sequences targeted are preferably of the same species as the subject being treated. Although in many instances, targets of a different species are also suitable, particularly those segments of the target RNA or gene that display greater than about 25% homology, greater than about 45% homology (i.e., identity of sequence residues), preferably greater than about 85% homology, still more preferably greater than about 95% homology, with the recipient's sequence. A preferable group is composed of des-A antisense oligos. Another preferred group is composed of non-fully desA oligos, where one or more adenosine bases are replaced with universal bases.

The oligo(s) of this invention reduce or inhibit gene expression of the target genes or mRNAs. This is generally attained by hybridization of the oligos to the gene of coding (sense) sequences of a targeted messenger RNA (mRNA). The agents are provided as a composition and various formulations, that decrease the levels of mRNA and encoded protein and/or cause changes in the growth characteristics or shapes of the treated cells. See, Milligan et al. J. Med. Chem. 36(14): 1923-1937 (1993); Helene, C. and Toulme, J., B. B. A 1049: 99-125 (1990); Cohen, J. S. D., Ed., Oligodeoxynucleotides as Antisense Inhibitors of Gene Expression, CRC Press, Boca Raton, Fla. (1987), the relevant portions of all of which are hereby incorporated in their entireties by reference. The mRNA sequence of a target may be derived from the nucleotide sequence of the corresponding gene or from the protein. For example, the sequence of the genomic human adenosine A₁ receptor and that of the rat and human adenosine A₃ receptors are known. See, U.S. Pat. No. 5,320,962; Zhou, F., et al., P. N. A. S. (USA) 89: 7432 (1992); Jacobson, M. A., et al., U.K. Patent Appl.; 93/04582.1. The sequence of the adenosine A_{2b} receptor gene is also known. See, Salvatore, C. A., Luneau, C. J., Johnson, R. G. and Jacobson, M., Genomics (1995), the relevant portion of all of which are hereby incorporated in their entireties by reference. The sequences of many of the exemplary target genes are also known. See, GENBANK database. The sequences of those genes whose sequences are not yet available may be obtained by isolating the target segments applying technology known in the art. Once the sequence of the gene, its RNA and/or the protein are known, an oligos may be produced according to this invention as described above to reduce the production of the targeted protein in accordance with standard techniques. The oligo of this patent clearly encompass sense and anti-sense, and double stranded sequences of DNA and RNA origin, as well as their combinations, analogues and salts.

In one aspect of this invention, the oligo has a sequence which specifically binds to a portion or segment of an mRNA molecule which encodes a protein associated with a disease or condition, for example, which is associated with airway and/or lung inflammation, allergy(ies), asthma, impeded respiration, cystic fibrosis (CF), Chronic Obstructive Pulmonary Diseases (COPD), allergic rhinitis (AR), Acute Respiratory Distress Syndrome (ARDS), pulmonary hypertension, lung inflammation, bronchitis, airway obstruction, infections including viral and bacterial infections, cancers, bronchoconstriction, and many others. One effect of this binding is to reduce or even prevent the translation of the corresponding mRNA and, thereby, reduce the available amount of target protein in the subject's lung. In one preferred embodiment of this invention, the phosphodiester residues of the oligo are modified or substituted. Chemical analogs of oligos with modified or substituted phosphodiester residues, e.g. to the methylphosphonate, the phosphotriester, the phosphorothioate, the phosphorodithioate, or the phosphoramidate, which increase the in vivo stability of the oligo are particularly preferred. The naturally occurring phosphodiester linkages of oligos are susceptible to some degree of degradation by cellular nucleases. Many of the residues proposed herein, on the contrary, are highly resistant to nuclease degradation. See Milligan et al., and Cohen, J. S. D., supra. In another preferred embodiment of the invention, the oligos may be protected from degradation by adding a “3′-end cap” by which nuclease-resistant linkages are substituted for phosphodiester linkages at the 3′ end of the oligo. See, Tidd, D. M. and Warenius, H. M., Be. J. Cancer 60: 343-350 (1989); Shaw, J. P. et al., Nucleic Acids Res. 19: 747-750 (1991), the relevant section of all of which are incorporated in their entireties by reference. Phosphoramidates, phosphorothioates, and methylphosphonate linkages all function adequately in this manner for the purposes of this invention. The more extensive the modification of the phosphodiester backbone the more stable the resulting agent, and in many instances the higher their RNA affinity and cellular permeation. See Milligan, et al., supra. The number of residues that may be modified or substituted will vary depending on the need, target, and route of administration, and may be from 1 to all the residues, to any number in between. Many different methods for replacing the entire phosphodiester backbone with novel linkages are known. See, Millikan et al, supra. Preferred backbone analogue residues include phosphorothioate, methylphosphonate, phosphotriester, thioformacetal, phosphorodithioate, phosphoramidate, formacetal boranophosphate, 3′-thioformacetal, 5′-thioether, carbonate, 5′-N-carbamate, sulfate, sulfonate, sulfamate, sulfonamide, sulfone, sulfite., sulfoxide, sulfide, hydroxylamine, methylene(methylimino) (MMI), and methyleneoxy(methylimino) (MOMI) residues. Phosphorothioate and methylphosphonate-modified oligos are particularly preferred due to their availability through automated oligo synthesis. See, Millikan et al, supra. Where appropriate, the agent of this invention may be administered in the form of their pharmaceutically acceptable salts, or as a mixture of the oligo and its salt. In another embodiment of this invention, a mixture of different oligos or their pharmaceutically acceptable slats is administered.

The oligo of this invention thus comprises an oligo(s) corresponding or being anti-sense to one (STAs), or two or more (MTA), wherein the target(s) comprise(s) genes or cDNA clones, mRNAs coding and non-coding regions, initiation codons of the genes, genomic flanking regions, intron-exon borders, 5′-end region, 3′-end region, regions within 2 to 10 nucleotides in length of the 5′-end or 3′-end, and the section straddling the coding and non-coding regions, precursor RNAs, poly-A segment, at least 4 contiguous mononucleotides of genes and RNAs encoding proteins known to be associated with one or more diseases or conditions, and then mixtures. The oligo(s) of this invention is(are) preferably designed to be anti-sense to a target gene(s) and/or mRNA(s) related in origin to the species to which it is(they are) to be administered. When treating humans, the oligos are preferably designed to be sense or anti-sense to a human gene or mRNA. The dry formulations of the invention encompass oligos which are sense or anti-sense to naturally occurring DNA and/or mRNA sequences, fragments thereof of up to a length of one (1) base less than the targeted sequence, preferably at least about 7 nucleotides long. Preferred are oligos having only up to about 0.02%, about 0.1%, about 1%, about 4% adenosine, about 5%, about 10%, about 15%, about 30% adenosine, although higher and lower amounts are also encompassed. In another preferred embodiment, the oligos have one or more mononucleotides replaced with so-called universal bases, which universal bases may pair up with the corresponding complementary mononucleotides.

The particle size of the agent of the invention altered to form a dry formulation, and is such as to permit inhalation or nasal administration or instillation into the lungs of a subject of a substantial amount of the material upon administration. In a first step the particle size desirably be less than about 1,000μ, preferably about 0.05μ, about 0.1μ, about 0.5μ to about 5μ, about 10μ, e.g. about 20μ, about 50μ, about 100μ. The particle size of the medicament or formulation may be then reduced by conventional means, for example, by milling or micronization. Generally, alteration of the particle size for the agent is produced by milling the dry pharmaceutical agent either alone or in combination with formulation ingredients to a suitable average particle size, typically where greater than about 80% of the particles are about 0.01μ to about 100μ, preferably, greater than about 85%, greater than about 90%, greater than about 95%, greater than about 98% of the particles are in the desired particle size range. Jet milling and fluid energy milling are often employed widely among the procedures to give the particle size of interest using known devices. Other than milling, the particle size may be altered to reduce by sieving, homogenization, granulation or any combination. These techniques are used either separately or in combination with themselves. Typically, milling, homogenization and granulation are applied followed by sieving to obtain the dry pharmaceutical agent with altered particle size. Although the agent is preferably formulated by itself, other embodiments contain ingredients such as a carrier or diluent, a preservative, a stabilizer, a powder flowability improving agent, a cohesiveness improving agent, a surfactant, other bioactive agents, a coloring agent, an aromatic agent, anti-oxidants, fillers, surfactants, volatile oils, dispersants, flavoring agents, buffering agents, RNA inactivating agents, bulking agents, propellants or preservatives. An example of the RNA inactivating agent is an enzyme, such as ribozyme.

In another embodiment, the particle size of the agent or formulation is reduced in a wet atmosphere. “Wet atmosphere” means that at least part of the procedure is conducted in a moist environment or by placing the agent in solution, suspension, or emulsion, either prior to, or subsequent to altering the particle size. For example, when precipitation or recrystallization to alter the particle size, the pharmaceutical agent may be dissolved in a suitable solution, suspension or emulsion, and heated to an appropriate temperature for a predetermined period of time to produce crystals. The solution along with the crystals may be cooled to a second temperature to anneal the crystals, e.g. by maintaining a second temperature for a pre-determined period of time. After cooling to room temperature, the recrystalization is completed, and crystals of the agent are allowed to grow sufficiently. The crystallized agent is obtained and is provided in a dry form for the next step. The alteration of the particle size may also be done through precipitation. This process can be conducted in the wet condition.

Spray freeze drying is useful in altering the particle size. By “spray freeze dried” herein is meant that the material is prepared by spray a process in which a homogeneous aqueous mixture of the pharmaceutical agent comprising an oligo, termed herein the “pre-spray dry formulation” is introduced via a nozzle (e.g. a two-fluid nozzle), spinning disk or an equivalent device to atomize the solution to form fine droplets. The aqueous solution is preferably a solution, although suspensions, emulsion, slurries or the like may be used as long as it is homogeneous to ensure uniform distribution of the material in the solution and ultimately in the powder formulation. Spraying the pharmaceutical agent through the nozzle results in the rapid forming of the atomized droplets to form particles. The particles may be collected, and the solvent removed, generally through sublimation or lyophilization in a vacuum. As discussed below, the particles may be annealed, i.e. the temperature raised, prior to drying. This produces a fine dry powder with particles of a specified size and characteristics that are more fully discussed below. Suitable spray drying methodologies are also described below.

The term “powder” means a composition that consists of finely dispersed solid particles that are relatively free flowing and capable of being readily dispersed in an inhalation device and subsequently inhaled by a patient so that the particles can reach the alveoli of the lung. Thus, the powder is “respirable” and suitable for pulmonary delivery. The term “dispersibility” means the degree to which a dry powder formulation can be dispersed (i.e. suspended) in a current of air so that the dispersed particles can be respired or inhaled into the lungs of a subject. Thus, a powder that is only 20% dispersible means that only 20% of the mass of particles can be suspended for inhalation into the lungs. The spray dried powders may be characterized on the basis of a number of parameters, including, but not limited to, the average particle size, the range of particle sizes, the fine powder fraction (FPF), the emitted dose, the average particle density, and the mass median aerodynamic diameter (MMAD).

In a preferred embodiment, the spray dried powder formulation of this invention is characterized on the basis of the proportion of particles of a certain average particle size. The average particle size generally ranges from about 0.05μ, about 0.1μ, about 0.5μ, about 1μ, about 1.5μ, about 5μ, about 8μ, about 10μ to about 15μ, about 20μ, about 30μ, about 50μ, about 80μin diameter. A strongly preferred average particle size is about 0.11 to about 5μ. The average particle size of the powder may be measured as mass mean diameter (MMF) by convention techniques. The term “about” means that the numerical values indicated may vary by about 10%. In another preferred embodiment, the dry powder formulation of this invention may be characterized on the basis of its fine particle fraction (FPF), a measure of the aerosol performance of a powder, where the higher the fraction, the better. In this patent, the “FPF” is defined as a powder with an aerodynamic mass median diameter of less than about 6.8 micron, as determined using a multiple-stage liquid impinger with a glass throat (MLSI, Astra, Copley Instrument, Nottingham, UK) through a dry powder inhaler (Dryhalter™, Dura Pharmaceuticals). Accordingly, the spray-dried powder of the invention has preferably a FPF of at least about 10%, at least about 20%, at least about 30%, with some systems enabling very high FPFs of the order of 40 to 50%. The dry powder formulation of the invention may be similarly characterized on the basis of particle density. In a preferred embodiment, the particles have a tap density of less than about 0.8 g/cm³, less than about 0.4 g/cm³, less than about 0.1 g/cm³. The tap density of dry powder particles, a standard measure of the envelope mass density, may be measured using a GeoPyc™ (Micrometrics Instruments Corp). The “envelope mass density” of an isotropic particle is given by the mass of the particle divided by the minimum sphere envelope volume within which it can be enclosed. In another preferred embodiment, the aerodynamic particle size of the dry powder formulation is characterized as is generally outlined in the Examples section. The mass median aerodynamic diameter (MMAD) of the particles may be evaluated similarly using techniques that are well known in the art. The particles may be characterized on the basis of their general morphology as well. The particles made by the processes of the invention may be spherical and porous, although other shapes and characteristics are also suitable. The term “dry” means that the formulation has a moisture content such that the particles are readily dispersible in a dry powder inhalation device to form an aerosol or spray. This moisture content is generally below about 15% w/w, less than about 10% w/w less than about 5% w/w, less than about 2.5% w/w, less than about 1% w/w, less than about 0.5% w/w in water. In addition, the dry powder formulations of substantially bioactive oligos, given that, as is known for many dry powder formulations, some percentage of the material in the powder may be damaged,, resulting in the reduction of the pulmonary delivery dose and in loss of activity. Accordingly, a preferred embodiment provides a dry powder formulation that has at least about 70% active oligo (i.e. the % of active oligo), at least about 80% active oligo, and at least about 90% active oligo, at least 95% active oligo. The measurement of total oligo present will depend on the oligo, and generally will be done as is known in the art, and may be attained through activity assays, etc. In spray drying, the individual stress event might arise due to atomization (shear stress and air-liquid interfacial stress), cold denaturation, freezing (ice-water interfacial stress and shear stress), and dehydration. Some studies have been devoted to understanding the effect of lyophilization on oligo stability using cryoprotectants against freezing destabilization, and lyoprotectants against dehydration and long-term storage destabilization. The mechanism of cryoprotectant molecules, e.g. sugars, amino acids, polyols, etc., preferentially excluded from contact with the oligo molecules has been widely used to provide oligo stabilization in the highly concentrated unfrozen liquid associated with ice crystallization.

The dry powder formulations comprising an oligo may preferably contain excipients. “Excipients” or “protectants” (including cryoprotectants and lyoprotectants) generally refer to compounds or materials that are added to ensure or increase the stability of the oligo, for long-term stability and flowability of the powder product and/or add bulk and improve the reproducible dispersing of the formulation. Suitable excipients are generally relatively free flowing particulate solids, do not thicken or polymerize upon contact with water, are basically innocuous when inhaled by a patient and do not significantly interact with the oligo in a manner that alters its biological activity. Suitable excipients include, but are not limited to, proteins such as human and bovine serum albumin, gelatin, immunoglobulins, carbohydrates including monosaccharides (galactose, D-mannose, sorbose, etc.), disaccharides (lactose, trehalose, sucrose, etc.), cyclodextrins, and polysaccharides (raffinose, maltodextrins, dextrans, etc.); an amino acid such as monosodium glutamate, glycine, alanine, arginine or histidine, as well as hydrophobic amino acids (tryptophan, tyrosine, leucine, phenylalanine, etc.); a methylamine such as betaine; an excipient salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; surfactants; and combinations thereof. Preferred excipients are trehalose, sucrose, sorbitol, salts such as sodium chloride, and lactose, or combination thereof. When excipients are used, they are used generally in amounts ranging from about 1%, about 5%, about 10%, to about 20%, about 30%, about 50%, about 100% w/w agent, depending on whether they are used for co-processing or as carriers. In yet another preferred embodiment, the dry powder formulations of in this invention does not contain substantial amounts of excipients, i.e. it is substantially free of excipients. “Substantially free” in this case means that the formulation contains less than about 10%, less than about 5%, less than about 3%, less than about 2%, less than about 1% w/w ingredients other than the agent and residual water. Generally, for the purposes of this invention, excipients do not include solvents, buffers or salts. Thus, preferred embodiments utilize spray dry formulations (prior to the addition of bulking agent, discussed below) that consist of the oligo as the major component, with small amounts of buffers, salts and residual water. In one embodiment, the spray dry process comprises an annealing step, where the temperature is raised prior to drying, as is more fully outlined below. In another preferred embodiment, the pre-spray dry formulation, i.e. the solution formulation used in the spray dry process, comprises the agent in water, with only negligible amounts of buffers or other compounds present. In some embodiments, the pre spray dried formulations containing little or no excipient may not be highly stable over long periods, and thus it is desirable to perform the spray drying process within a reasonable short time after producing the pre-spray dried formulation. While the pre-spray dried formulation utilizing little or no excipient may not be highly stable, the dry powder made from it may be surprisingly stable and highly dispersible, as is shown in the Examples. In still another preferred embodiment, the pharmaceutical agents that are spray dried to form the formulations of the invention comprise the DNA or RNA in a buffer, and may or may not additionally contain some salts. The pH of the buffer is generally chosen to stabilize the active agent, and generally will be present at almost physiological pH, although some oligo, such as SEQ ID NO. 1, may be stable at a wider range of pHs, including acidic pH. Thus, preferred pH for the pre-spray drying formulation is about 1, about 3, about 5, about 6.5 to, about 7, about 8, about 9, about 10. As will be appreciated by those in the art, there are numerous suitable buffers that may be used. Suitable buffers include, but are not limited to, sodium acetate, sodium phosphate, sodium citrate, sodium succinate, and ammonium bicarbonate and carbonate among others, as well as salts of the cations. Buffers are used generally at about 0.05 mM, about 1 mM, about 2 mM, about 10 mM, about 50 mM to about 200 mM, about 1M, about 2M. When the formulation is spray dried the reagent may be placed in a solvent(s), that may or may not additionally contain salts. The pH of the solvent will vary with the oligo, as will be appreciated by those in the art, with pharmaceutically acceptable solvents preferred. Suitable pH ranges and molarities are as outlined above for buffers. As will be appreciated by those in the art, there are a large number of suitable solvents that may be used, among which included but not limited to, acids including acetic and citric acid, and alcohols such as ethanol. When water, buffers or solvents are used, they may additionally contain salts, generally present at about 0.05 mM, about 0.1 mM, about 0.5 mM, about 1 mM to about 250 mM, about 500 mM, about 1M, about 2M, about 2.5M. Suitable salts include, but are not limited to, NaCl. The dry powder formulation of the invention may be generally substantially free of “penetration enhancers”, or surface active compounds that promote the penetration of a drug through a mucosal membrane or lining, and are generally used intranasally, intrarectally, and intravaginally, among other routes. The use of strong penetration enhancers in the lungs however, may be undesirable in some instances as the sensitive and fragile epithelial blood barrier in the lung may be affected by surface active compounds, such as detergents. The dry powder formulation of the invention is, in general, readily absorbed in the lungs without the need to employ penetration enhancers. The dry powder formulation of the invention is generally substantially free of microsphere-forming polymers. See, for example, WO 97/44013, U.S. Pat. No. 5,019,400. That is, the powder formulation of the invention generally comprises DNA and/or RNA and excipient, and does not require the use of polymers for structural purposes.

Furthermore, the dry powder formulation of the invention is generally stable. “Stability” may mean stability in retention of biological activity, and retention of dispersibility over time. The dry powder formulation of the invention is said to retain biological activity over time when it retains its physical and chemical stability and integrity upon storage. Losses in biological activity are generally due to degradation, and oxidation. As will be appreciated by those in the art, there may be an initial loss of biological activity as a result of spray drying, due to the temperatures used in the process. Once this step has occurred, further loss of activity should be minimized; that is, stability in this context is measured from the time the powder is made, rather than before the powder is made. The dry powder formulation of the invention generally retains dispersibility over time that is, the powder minimally aggregates, cakes or clumps over time. This may be quantified by retention of a high FPF over time. The dry powder formulation of the invention may be produced, as follows. Generally, the pharmaceutical agent comprising an oligo is made by any of many methods known in the art. Initially, the agent may be placed for stability in a liquid or solid composition, or subjected to size modification by itself. For spray drying, a liquid formulation is generally subjected to diafiltration and/or ultrafiltration, for buffer exchange (or removal) and/or for concentration, as is known in the art. The pre-spray dry formulation generally comprises about 0.5 mg/ml, about 5 mg/ml, about 10 mg/ml, about 20 mg/ml to about 40 mg/ml, about 60 mg/ml, about 70 mg/ml, about 100 mg/ml, about 500 agent. The use of buffers and excipients, if present, is done at standard concentrations that are briefly discussed above. The pre-spray formulation may be then spray dried by first dispersing into hot air or gas, or by spraying into a cold stream of supercritical freezing fluid such as liquid or gas. The pre-spray dry formulation may be also atomized as is known in the art, for example via a two-fluid nozzle or an ultrasonic nozzle, using filtered pressurized air into drying gas. Conventional spray drying equipment may be employed (e.g. Buchi, Niro Yamato, Okawara, Kakoki and the like). When a cold fluid is used, it is generally preferable to slightly heat the nozzle, for example by wrapping the nozzle with heating tape, to prevent the nozzle head from freezing. The pre-spray formulation may be atomized into a cold fluid. Generally, temperatures ranging from about −200° C. to about −100° C., about −80° C. are used, with about −200° C. being preferred (e.g. liquid nitrogen at −196° C.). The fluid may be a liquid such as liquid nitrogen or other inert fluids, or a gas such as air that is cooled. Dry ice in ethanol is generally suitable, as is the use of super critical fluids. In some embodiments it is preferred to stir the liquid as the atomization process occurs, although this may not be required.

Supercritical fluid processes may be used for altering the particle size of the agent. Supercritical fluid processes involve precipitation by rapid expansion of supercritical solvents, gas anti-solvent processes, and precipitation from gas-saturated solvents. A supercritical fluid is applied at a temperature and pressure that are greater than its critical temperature (T_{c}) and critical pressure (P_{c}), or compressed fluids in a liquid state. It is known that at near-critical temperatures, large variations in fluid density and transport properties from gas-like to liquid-like can result from relatively moderate pressure changes around the critical pressure (0.9-1.5 P_{c}) While liquids are nearly incompressible and have low diffusivity, gases have higher diffusivity and low solvent power. Supercritical fluids can be made to possess an optimum combination of these properties. The high compressibility of supercritical fluids (implying that large changes in fluid density can be brought about by relatively small changes in pressure, making solvent power highly controllable) coupled with their liquid-like solvent power and better-than-liquid transport properties (higher diffusivity, lower viscosity and lower surface tension compared with liquids), provide a means for controlling mass transfer (mixing) between the solvent containing the solutes (such as a drug) and the supercritical fluid.

The two processes that use supercritical fluids for particle formation and that have received attention in the recent past are: (1) Rapid Expansion of Supercritical Solutions (RESS) (Tom, J. W. Debenedetti, P. G., 1991, The formation of bioerodible polymeric microspheres and microparticles by rapid expansion of supercritical solutions. BioTechnol. Prog. 7:403-411), and (2) Gas Anti-Solvent (GAS) Recrystallization (Gallagher, P. M., Coffey, M. P., Krukonis, V. J., and Klasutis, N., 1989, GAS antisolvent recrystallization: new process to recrystallize compounds in soluble and supercritical fluids. Am. Chem. Sypm. Ser., No. 406; Yeo et al. (1993); U.S. Pat. No. 5,360,478 to Krukonis et al.; U.S. Pat. No. 5,389,263 to Gallagher et al.). In the RESS process, a solute (from which the particles are formed) is first solubilized in supercritical CO₂ to form a solution. The solution is then, for example, sprayed through a nozzle into a lower pressure gaseous medium. Expansion of the solution across this nozzle at supersonic velocities causes rapid depressurization of the solution. This rapid expansion and reduction in CO₂ density and solvent power leads to supersaturation of the solution and subsequent recrystallization of virtually contaminant-free particles. The RESS process, however, may not be suited for particle formation from polar compounds because such compounds, which include drugs, exhibit little solubility in supercritical CO₂ Cosolvents (e.g., methanol) may be added to CO₂ to enhance solubility of polar compounds; this, however, affects product purity and the otherwise environmentally benign nature of the RESS process. The RESS process also suffers from operational and scale-up problems associated with nozzle plugging due to particle accumulation in the nozzle and to freezing of CO₂ caused by the Joule-Thompson effect accompanying the large pressure drop.

In the GAS process, a solute of interest (typically a drug) that is in solution or is dissolved in a conventional solvent to form a solution is sprayed, typically through conventional spray nozzles, such as an orifice or capillary tube, into supercritical CO₂ which diffuses into the spray droplets causing expansion of the solvent. Because the CO₂-expanded solvent has a lower solubilizing capacity than pure solvent, the mixture can become highly supersaturated and the solute is forced to precipitate or crystallize. The GAS process enjoys many advantages over the RESS process. The advantages include higher solute loading (throughput), flexibility of solvent choice, and fewer operational problems in comparison to the RESS process. In comparison to other conventional techniques, the GAS technique is more flexible in the setting of its process parameters, and has the potential to recycle many components, and is therefore more environmentally acceptable. Moreover, the high pressure used in this process (up to 2,500 psig) can also potentially provide a sterilizing medium for processed drug particles; however, for this process to be viable, the selected supercritical fluid should be at least partially miscible with the organic solvent, and the solute should be preferably insoluble in the supercritical fluid.

Gallagher et al. (1989) teach the use of supercritical CO₂ to expand a batch volume of a solution of nitroguanadine and recrystallize particles of the dissolved solute. Subsequent studies disclosed by Yeo et al. (1993) used laser-drilled, 25-30 μm capillary nozzles for spraying an organic solution into CO₂. Use of 100 μm and 151 μm capillary nozzles also has been reported (Dixon, D. J. and Johnston, K. P., 1993, Formation of microporous polymer fibers and oriented fibrils by precipitation with a compressed fluid antisolvent. J. App. Polymer Sci. 50:1929-1942; Dixon, D. G., Luna-Barcenas, G., and Johnson K. P., 1994, Microcellular microspheres and microballoons by precipitation with a vapour-liquid compressed fluid antisolvent. Polymer 35:3998-4005). Examples of solvents are selected from carbon dioxide (CO₂), nitrogen (N₂), Helium (He), oxygen (O₂), ethane, ethylene, ethylene, ethane, methanol, ethanol, trifluoromethane, nitrous oxide, nitrogen dioxide, fluoroform (CHF₃), dimethyl ether, propane, butane, isobutanes, propylene, chlorotrifluormethane (CClF₃), sulfur hexafluoride (SF₆), bromotrifluoromethane (CBrF₃), chlorodifluoromethane (CHClF₂), hexafluoroethane, carbon tetrafluoride carbon dioxide, 1,1,1,2-tetrafluoroethane, 1,1,1,2,3,3,3-heptafluoropropane, xenon, acetonitrile, dimethylsulfoxide (DMSO), dimethylformamide (DMF), and mixtures of two or more thereof.

The atomization conditions, including the atomization gas flow rate, atomization gas pressure, liquid flow rate, etc., are generally controlled to produce liquid droplets in accordance with dₚ=d_{d}(Cv)^{1/3}. Once the droplets are produced, they may be dried, that is, the water removed, leaving the oligo, any excipients, and residual buffers, solvents or salts. This is may be done in a variety of ways known in the art. That is, techniques that may be used for traditional lyophilization, i.e. freezing as a cake rather than as droplets, may be used. Generally, and preferably, a vacuum is applied, which may be applied at about the temperature used for freezing. As shown in the examples, however, it may be possible to relieve some of the freezing stress on the oligo by raising the temperature of the frozen particles slightly prior to or during the application of the vacuum. This process, termed “annealing”, has been shown to reduce oligo inactivation. This may be done in one or more steps by increasing the temperature one or more times either before or during the drying step of the vacuum. Preferred embodiments utilize at least two thermal increases. The particles are incubated, preferably for a period of time, sufficient for thermal equilibrium to be reached, i.e., depending on sample size and efficiency of heat exchange one to several hours is generally sufficient, prior to the application of vacuum. The vacuum is applied and another annealing step is done. This is particularly desirable when double stranded oligos are formulated. The particles may be lyophilized for a period of time sufficient to remove the majority of the water in the particles, the actual period of time depending on the temperature, strength of the vacuum, size of the sample, etc. Generally, the particles are lyophilized to a dryness of about 0.5%, about 1% to about 5%, about 10% remaining water. A secondary drying step under lyophilization may be conducted to remove additional water. This is generally done at temperatures from about 0° C., about 10° C. to about 20° C., about 30° C., about 40° C., about 50° C.

The powders are collected using conventional techniques, and bulking agents, if desirable, are added.

Once made, the dry powder formulations in the invention are capable of being readily dispersed by an inhalation device and subsequently inhaled by a patient so that the particles are able to penetrate into the alveolar regions of the lungs of the patient. Thus, the powders of the invention are formulated into unit dosages comprising therapeutically effective amounts of oligo, and used to deliver pharmaceutical agent comprising an oligo to a patient, for example, for the treatment of any number of disorders that are associated with the particular oligo.

The dry powder formulations comprising an oligo to be used in the therapy will be formulated and dosed in a fashion consistent with good medical practice, taking into account, for example, the type of disorder being treated, the clinical condition of the individual patient (especially the side effects of treatment with the oligo), whether the oligo is administered for preventative or therapeutic purposes, the concentration of the oligo in the dosage, previous therapy, the patient's clinical history and response to the oligo, the method of administration, the scheduling of administration, the discretion of the attending physician, and other factors known to practitioners. The “effective amount” or “therapeutically effective amount” of the oligo for purposes herein will depend on the identity of the oligo and is thus determined by such considerations and is an amount that increases and maintains the relevant, favorable biological response of the mammal. The oligo is suitably administered to the patient at one time or over a series of treatments and may be administered to the patient at any time from diagnosis onwards.

Thus, this invention provides spray-dried dry powder formulations comprising an oligo in unit dosages. A “unit dosage” as discussed herein means a unit dosage receptacle containing a therapeutically effective amount of a spray dried oligo. The dosage receptacle is one that fits within a suitable inhalation device to allow for the aerosolization of the dry powder formulation by dispersion into a gas stream to form an aerosol. These can be capsules, foil pouches, vials, etc. The container may be formed from any number of different materials, including plastic, glass, foil, etc. The container generally holds the spray-dried powder, and includes directions for use. The unit dosage containers may be associated with inhalers that will deliver the powder to the patient. These inhalers may optionally have chambers into which the powder is dispersed, suitable for inhalation by a patient.

Additionally, the dry powder formulations of the invention may be further formulated in other ways, for example, in the preparation of sustained release compositions, for example for implants, patches, etc. Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman et al., Biopolymers, 22, 547-556 [1983]), poly(2-hydroxyethyl methacrylate) (Langer et al., J. Biomed. Mater. Res., 15: 167-277 (1981), and Langer, Chem. Tech., 12: 98-105 [1982]), ethylene vinyl acetate (Langer et al., supra) or poly-D-(-)-3-hydroxybutyric acid (EP 133,988). Sustained-release compositions also include liposomally entrapped proteins. Liposomes containing proteins are prepared by methods known per se: DE 3,218,121; Epstein et al., Proc. Natl. Acad. Sci. U.S.A., 82: 3688-3692 (1985); Hwang et al., Proc. Natl. Acad. Sci. U.S.A., 77: 4030-4034 (1980); EP 52,322; EP 36,676; EP 88,046; EP 143,949; EP 142,641; Japanese Patent Appln. 83-118008; U.S. Pat. Nos. 4,485,045 and 4,544,545; and EP 102,324. Ordinarily, the liposomes are of the small (from or about 200 to 800 Angstroms) unilamellar type in which the lipid content is greater than about 30 mol. percent cholesterol, the selected proportion being adjusted for the optimal therapy.

In a preferred embodiment, the dry powder formulations in the invention are not inhaled; rather they are injected as dry powders, using relatively new injection devices and methodologies for injecting powders. In this embodiment, the dispersibility and respirability of the powder is not important, and the particle size may be larger, for example in the about 20 to about 70 micron range.

It should also be noted that the dry powder formulations in the invention may be reconstituted for injection as well. That is, the powders of the invention show good stability, and thus in some embodiments they can be reconstituted into liquid form using a diluent and used in non-pulmonary routes of administration, for example, via injection (subcutaneously, intravenously, etc.). In this embodiment, any number of known diluents can be used, as will be appreciated by those in the art, including physiological saline, other buffers, salts, etc. Alternatively, it is also possible to reconstitute the powder and use it to form liquid aerosols for pulmonary delivery.

As used herein, the term “treating” refers to therapeutic, prophylactic and preventative treatments in need of treatment include those already with the disorder as well as those prone to having the disorder or that are diagnosed with the disorder or in whose the disorder is to be prevented. Consecutive treatment or administration refers to treatment on at least a daily basis without interruption in treatment by one or more days. Intermittent treatment or administration, or treatment or administration in an intermittent fashion, refers to treatment that is not consecutive, but rather cyclic in nature. The treatment regime herein may be consecutive or intermittent or any other suitable mode. In addition, the term “treating” includes management of a particular disorder, as in the management of hyperglycemic disorders and obesity.

The particle size of the dry powder formulation is selected greater than about 80%, about 85%, about 90%, about 95% oligo particles of about 0.1μ, about 0.5μ, about 1μ to about 5μ, about 10μ, about 20μ, about 40μ, about 60μ, about 80μ, about 100μ. To obtain the dry powder formulation having the preferable particle size, several conventional methods may be employed, for example, sieving, lyophilization, spray-lyophilization, spray drying, spray freeze-drying, etc. The use of filters employed for sieving is known to the skilled artisan. The method of the invention may comprise selecting the particle size not only in a dry atmosphere but also a moist or liquid atmosphere. The “wet” atmosphere refers to the fact that at least part of the method is conducted under moist conditions or in a solution, suspension, or emulsion. Accordingly, the pharmaceutical agent may be placed under moist conditions or in a solution, suspension, or emulsion, and the particle size alteration and selection may be conducted in a single or multiple step(s). The alteration and selection of particle size may be conducted preferably by spray drying under conditions effective to attain the desired particle size. The method of this invention may further comprise storing the dry powder formulation obtained under controlled conditions, e.g. of temperature, humidity, light, pressure or other conditions that do not significantly alter the flowability or activity of the agent. Stability upon storing may be measured at a pre-selected temperature for a specialized period of time The stability of the formulation will depend on the listed storage conditions, turnover of product, etc. Generally, for rapid screening, a matrix of conditions are tested, for example about 15° C., about 20° C., about 25° C., about 40° C., for periods of about 1 month, about 3 months, about 4 months, about 12 months, about 24 months, about 36 months. These tests are usually done at about 60% relative humidity (rh) according to ICH Guidelines Under the method of the invention the agent generally loses less than about 30%, less than about 20%, less than about 10% of their biological activity over about 24 months to 36 months. When dispersibility is being evaluated, the powder of the invention generally lose less than about 30%, less than about 20% of its FPF.

In a preferred embodiment, the spray dried powder of the invention may be later combined with formulation ingredients, such as bulking agents or carriers, which are used to reduce the concentration of the pharmaceutical agent in the powder being delivered to a patient; that is, it may be desirable to have larger volumes of material per unit dose. Bulking agents may also be used to improve the dispersibility of the powder within a dispersion device, and/or to improve the handling characteristics of the powder. This is distinguishable from the use of bulking agents or carriers during the spray drying process. Suitable bulking agents are generally used to avoid water absorption and include, but are not limited to, lactose and mannitol. Accordingly, bulking agents such as lactose, if added, may be added in varying ratios, with from about 1:about 99, about 1:about 10, about 1:about 20, about 1:about 5 bulking agent to about 1:99 being preferred, and from about 1:5 to about 5:1 being more preferred, and from about 1:10 to about 1:20 being especially preferred. The dry powder formulation of the invention may be prepared alone or with other drugs. That is, combinations of a therapeutic oligos and the therapeutic agents may be spray dried, or they may be spray dried separately and combined, or one component may be spray dried and the other may not. The agents may be also administrated separately. The combination of drugs will depend on the disorders for which they are given, as will be appreciated by those in the art. The dry powder formulation of the invention may comprise also formulation ingredients, preservatives, detergents, surfactants, antioxidants, etc., that are generally known in the art. The dry powder formulations of the invention may be administered by any means that transports the agent to the airways, and may be administered to the airways by any suitable means, but is preferably administered through the respiratory system as a respirable formulation, more preferably in the form of a dry powder aerosol or spray comprising respirable particles that, in turn, comprise the agent The dry powder formulation of this invention comprises preferably particles of respirable size, preferably of a size sufficiently small to pass, upon inhalation into the bronchi and alveoli of the lungs. In general, particles ranging from about 0.05μ to about 100μ in diameter are respirable. Suitable and preferred particle diameters are about 0.05μ, about 0.1μ to about 5μ, about 10μ for inhalation. Particles of non-respirable size, of considerably larger diameter, which are included in the respirable formulation tend to deposit in the throat and may be swallowed. Accordingly, it is desirable to minimize the quantity of non-respirable particles in the formulation. For nasal administration, a particle size in the range of about 8μ, about 10μ, about 20μ, about 30μ, about 50μ, about 60μ, about 80μ, about 100μ are preferred to ensure their retention in the nasal cavity.

In another preferred embodiment, the dry powder formulation in this invention may comprise the dry pharmaceutical agent comprising the oligo(s), and one or more surfactant(s). Suitable surfactants or surfactant components that either are effective for treating respiratory ailments or enhance the uptake of the oligos, include synthetic and natural as well as full and truncated forms of surfactant proteins, e.g. surfactant protein A, surfactant protein B, surfactant protein C, surfactant protein D and surfactant protein E, phospholipids such as di-saturated phosphatidylcholine (other than dipalmitoyl), dipalmitoylphosphatidylcholine, phosphatidylcholine, phosphatidylglycerol, phosphatidylinositol, phosphatidylethanolamine, phosphatidylserine, other surfactants such as lysophosphatidylethanolamine, lysophosphatidylcholine, palmitoyl-lysophosphatidylcholine, phosphatidic acid, glycero-3-phosphocholine, ubiquinones, dehydroepiandrosterone, dolichols, sulfatidic acid, glycerol-3-phosphate, dihydroxyacetone phosphate, glycerol, dihydroxyacetone, palmitate, cytidine diphosphate (CDP) diacylglycerol, CDP choline, choline, choline phosphate; natural and artificial lamelar bodies as natural surfactant carrier, other acids such as omega-3 fatty acids, polyenic acid, polyenoic acid, lecithin, palmitinic acid, polymers such as non-ionic block copolymers of ethylene or propylene oxides, polyoxypropylene, monomeric and polymeric polyoxyethylene, monomeric- and polymeric-poly(vinylamine) with dextran and/or alkanoyl side chains, Brij 35®, Triton X-100® and synthetic surfactants such as ALEC®, Exosurf®, Survan® and Atovaquone® , among others. These surfactants may be used either alone, as part of a multiple component surfactant, or as covalently bound additions to the 5′- and/or 3′-ends of the agents comprising the oligos. The dry powder formulation of the invention may be administered by any means which transports the agent and, if necessary, other therapeutic agents to the lung. The dry powder formulations disclosed herein may be administered to the lungs of a patient by any suitable means, but are preferably administered by inhalation of an aerosol or spray comprised of respirable particles of the dry powder formulations. Nasal and intrapulmonary administration and other routes described above are also contemplate. The particles of the formulation may optionally contain other therapeutic or diagnostic ingredients, such as analgesics e.g. Acetaminophen, Anilerdine, Aspirin, Buprenorphine, Butabital, Butorpphanol, Choline Salicylate, Codeine, Dezocine, Diclofenac, Diflunisal, Dihydrocodeine, Elcatoninin, Etodolac, Fenoprofen, Hydrocodone, Hydromorphone, Ibuprofen, Ketoprofen, Ketorolac, Levorphanol, Magnesium Salicylate, Meclofenamate, Mefenamic Acid, Meperidine, Methadone, Methotrimeprazine, Morphine, Nalbuphine, Naproxen, Opium, Oxycodone, Oxymorphone, Pentazocine, Phenobarbital, Propoxyphene, Salsalate, Sodium Salicylate, Tramadol and Narcotic analgesics in addition to those listed above. See, Mosby's Physician's GenRx. Anti-anxiety agents are also useful including Alprazolam, Bromazepam, Buspirone, Chlordiazepoxide, Chlormezanone, Clorazepate, Diazepam, Halazepam, Hydroxyzine, Ketaszolam, Lorazepam, Meprobamate, Oxazepam and Prazepam, among others. Anti-anxiety agents associated with mental depression, such as Chlordiazepoxide, Amitriptyline, Loxapine Maprotiline and Perphenazine, among others. Anti-inflammatory agents such as non-rheumatic Aspirin, Choline Salicylate, Diclofenac, Diflunisal, Etodolac, Fenoprofen, Floctafenine, Flurbiprofen, Ibuprofen, Indomethacin, Ketoprofen, Magnesium Salicylate, Meclofenamate, Mefenamic Acid, Nabumetone, Naproxen, Oxaprozin, Phenylbutazone, Piroxicam, Salsalate, Sodium Salicylate, Sulindac, Tenoxicam, Tiaprofenic Acid, Tolmetin, anti-inflammatories for ocular treatment such as Diclofenac, Flurbiprofen, Indomethacin, Ketorolac, Rimexolone (generally for post-operative treatment), anti-inflammatories for, non-infectious nasal applications such as Beclomethaxone, Budesonide, Dexamethasone, Flunisolide, Triamcinolone, and the like. Soporifics (anti-insomnia/sleep inducing agents) such as those utilized for treatment of insomnia, including Alprazolam, Bromazepam, Diazepam, Diphenhydramine, Doxylamine, Estazolam, Flurazepam, Halazepam, Ketazolam, Lorazepam, Nitrazepam, Prazepam Quazepam, Temazepam, Triazolam, Zolpidem and Sopiclone, among others. Sedatives including Diphenhydramine, Hydroxyzine, Methotrimeprazine, Promethazine, Propofol, Melatonin, Trimeprazine, and the like. Sedatives and agents used for treatment of petit mal and tremors, among other conditions, such as Amitriptyline HCl; Chlordiazepoxide, Amobarbital; Secobarbital, Aprobarbital, Butabarbital, Ethchiorvynol, Glutethimide, L-Tryptophan, Mephobarbital, MethoHexital Na, Midazolam HCl, Oxazepam, Pentobarbital Na, Phenobarbital, Secobarbital Na, Thiamylal Na, and many others. Agents used in the treatment of head trauma (Brain Injury/Ischemia), such as Enadoline HCl (e.g. for treatment of severe head injury; orphan status, Warner Lambert), cytoprotective agents, and agents for the treatment of menopause, menopausal symptoms (treatment), e.g. Ergotamine, Belladonna Alkaloids and Phenobarbital, for the treatment of menopausal vasomotor symptoms, e.g. Clonidine, Conjugated Estrogens and Medroxyprogesterone, Estradiol, Estradiol Cypionate, Estradiol Valerate, Estrogens, conjugated Estrogens, esterified Estrone, Estropipate, and Ethinyl Estradiol. Examples of agents for treatment of pre-menstrual syndrome (PMS) are Progesterone, Progestin, Gonadotrophic Releasing Hormone, Oral contraceptives, Danazol, Luprolide Acetate, Vitamin B6. Examples of agents for treatment of emotional/psychiatric treatments such as Tricyclic Antidepressants, including Amitriptyline HCl (Elavil), Amitriptyline HCl, Perphenazine (Triavil) and Doxepin HCl (Sinequan). Examples of tranquilizers, anti-depressants and anti-anxiety agents are Diazepam (Valium), Lorazepam (Ativan), Alprazolam (Xanax), SSRI's (selective Serotonin reuptake inhibitors), Fluoxetine HCl (Prozac), Sertaline HCl (Zoloft), Paroxetine HCl (Paxil), Fluvoxamine Maleate (Luvox), Venlafaxine HCl (Effexor), Serotonin, Serotonin Agonists (Fenfluramine), and other over the counter (OTC) medications.

The dry powder formulation of the invention may be produced with any device that generates a solid particulate medicament aerosol. Aerosol generators suitable for administering solid particulate medicaments produce respirable particles, as explained above, and generate a volume of dry powder aerosol or spray containing a pre-determined metered dose of the agent at a rate suitable for human or animal administration. One illustrative type of solid particulate aerosol or spray generator is a dry powder inhalator. Suitable formulations for administration by insufflation include finely comminuted powders that may be delivered by means of an insufflator or taken into the nasal cavity in the manner of a snuff. In the insufflator, the powder, e.g. a metered dose of the agent effective to carry out the treatments described herein, is contained in a capsule or a cartridge. These capsules or cartridges are typically made of gelatin or plastic, and may be pierced or opened in situ, and the powder delivered by air drawn through the device upon inhalation or by means of a manually-operated pump. The dry powder formulation employed in the insufflator may consist either solely of the agent or of a powder blend comprising the agent. The formulation typically comprises from about 0.01 to about 100% w/w agent/formulation.

Suitable amounts of powder for administration are about 1 ng, about 5 ng, about 10 ng, about 20 ng, to about 25 ng, about 30 ng, about 35 ng, about 50 ng, and the like. Other ingredients, and other amounts of the agent are also suitable within the confines of this invention. The formulation of the invention is provided also in various forms that are tailored for different methods of administration and routes of delivery. The formulations that are contemplated are, for example, a transdermal formulation also containing carrier(s) and other agents suitable for delivery through the skin, mouth, nose, vagina, anus, eyes, ears, and other body cavities, intradermally, as a sustained release formulation, intracranial, intrathecally, intravascularly, by inhalation, intrapulmonarily, into an organ, by implantation, including suppositories, cremes, gels, and the like, as is known in the art. In one particular formulation, the dry powder formulations are further suspended or dissolved in a solvent. In another, the carrier comprises a hydrophobic carrier, such as lipid particles or vesicles, including liposomes and micro crystals. The preparation of all of these formulations, as well as the ingredients to be utilized, are known in the art, and need not be further described here. In one particularly preferred embodiment of the vesicle formulation, the vesicles comprise liposomes containing the oligo. The lipid vesicles may comprise N-(1-[2,3-dioleoxyloxy]propyl)-N,N,N-trimethyl-ammonium methylsulfate as well as other lipids known in the art to provide suitable delivery of DNA to target cells. In one embodiment, the dry powder formulation comprises a respirable formulation, such as an aerosol. The dry powder formulation of the invention are provided in bulk, and in unit form, as well as in the form of an implant, further dissolved in a solution, suspension or emulsion, a capsule or cartridge, which may be openable or piercable as is known in the art.

A kit is also provided, which comprises a dry powder delivery device, and in separate containers, the dry powder formulation of the invention, and optionally other agents, and instructions for the use of the kit components. In one preferred embodiment, the delivery device comprises a dry powder inhalator that delivers single or multiple doses of the formulation. The single dose inhalator may be provided as a disposable kit that is sterilely pre-loaded with enough formulation for one application. The inhalator may be adapted to pierce or open capsules, blisters or cartridges, and the formulation in a piercable or openable capsule(s), blister(s) or cartridge(s). The kit may optionally also comprise in a separate container other therapeutic compounds, anti-oxidants, flavoring and coloring agents, fillers, volatile oils, buffering agents, dispersants, surfactants, cell internalized or up-taken agents, RNA inactivating agents, anti-oxidants, flavoring agents, bulking agents, propellants, co-solvents, and preservatives, among other suitable additives.

When a sense oligo is employed, it may be associated with infection or other cancerous target, intended as a vaccine for expression of a pre-determined sequence. When administered as an anti-sense oligo, in the treatment of a disease or condition associated with the mRNA corresponding to at least one target gene(s), to genomic flanking regions, initiation codon, intron-exon borders and the like, or the entire sequence of precursor RNAs, including non-coding RNA segments, the 5′-end and the 3′-end, e.g. poly-A segment and oligos targeted to the section bridging coding and non-coding regions, and RNA regions encoding proteins, by administration to a subject afflicted with the disease or condition of an amount of the oligo effective to reduce the production or availability, or to increase the degradation by the subject of at least one of the target mRNAs. Typically, the dry powder formulation is administered in an amount effective to reduce the production or availability, or to increase the degradation of one or more target mRNAs. Optionally, the dry powder formulation is administered directly to the lung(s) of the subject, preferably as a dry respirable aerosol or spray. Although an artisan will know how to titrate the amount of dry powder formulation to be administered by the weight of the subjected being treated, the agent is preferably administered in an amount effective to attain an intracellular concentration of about 0.05 to about 10 μM oligo, preferably in an amount effective to attain an intracellular concentration of up to about 5 μM oligo. The dry powder formulation of the invention may be delivered in one of many ways. Accordingly, this invention provides methods for delivering the dry powder formulation to a target tissue or organ, comprising administering to a subject an effective amount of the dry powder formulation. For example, administration are done by a transdermal or systemic route, and more specifically orally, intracavitarily, intranasally, intraanally, intravaginally, transdermally, intrabucally, intravenously, subcutaneously, intramuscularly, intratumorously, into a gland, by implantation, intradermally, and many other routes of administration. The dry powder formulation may be, in addition, an implant, slow release, transdermal release, sustained release, and coated with one or more macromolecules to avoid destruction of the agent prior to reaching the selected target. The subject that may be treated by this agent are varied, and include humans and other animals in general, and in particular vertebrates, and amongst these mammals, and more specifically humans, and small and large, wild and domesticated, marine and farm animals, and preferably humans and domesticated and farm animals. In one aspect of the invention, at least one of the target mRNAs and the subject are of the same species, and in a preferred case they are of human origin. However, since in one embodiment mismatched nucleotides are replaced, mismatched species may also be utilized.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes.

### EXAMPLES

All references cited herein are incorporated by reference in their entirety. In these examples, μM means micromolar, mM means milimolar, ml means milliliters, μ or micron means micrometers, mm means millimeters, cm means centimeters, ° C. means degrees Celsius, μg means micrograms, mg means milligrams, g means grams, kg means kilograms, M means molar, and h means hours.

### Example 1

### Spray Drying of Oligo (1) & Particle Size Determination

Two batches of oligo were prepared for spray drying. One gram of 21 bp oligo (SEQ ID NO: 1) was dissolved to 100 ml of water to produce a 1% solution. Likewise, 2 grams of the oligo were dissolved to 200 ml of water to produce 1% solution. The solution was spray-dried with a B-191 Mini Spray-Drier (Buchi, Flawil, Switzerland) under the following conditions: inlet temperature=70° C., outlet temperature=50° C., aspirator=100%, pump=10%, nitrogen flow=40 mbar, spray flow=600 L/hr.

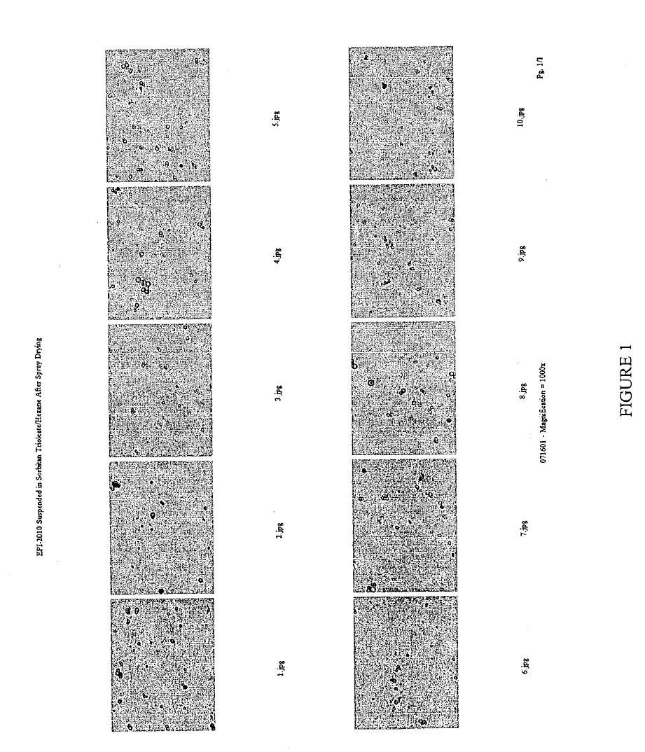

The spray dried product from each batch was suspended in hexane. Span85 was used as a surfactant to keep the particles from agglomerating and the dispersions were sonicated with cooling for 1-3 minutes for complete dispersion. The dispersed solutions were tested on a Malvern Mastersizer X with a Small Volume Sampler (SVS) attachment. The two batches of spray dried material were found to have mean particle sizes of 4.07±0.045 μm and 3.27±0.131 μm. Visual examination of unsonicated dispersions of each batch confirmed that spray drying produced small respirable size particles. The % respirable particles (% particles below 5 μm) ranged from 86.6% to 90.8% and the mean particle size was 2.96±1.89 μm and 2.51±1.82 μm for each batch, respectively. A photograph of spray dried material is presented in FIG. 1.

### Example 2

### Stability Study of Lyophilized Oligo Formulations

This study was conducted to assess the stability of lyophilized prototype formulations containing 12 mg/vial of pure oligonucleotide (oligo) of SEQ ID NO: 1. The stability of the formulations was assessed as a function of temperature of 40±2° C., preparation solvent of water for injection and phosphate buffer pH 7, storage orientation of the reconstituted product of 24 hours of upright and inverted room temperature storage following reconstitution. The following parameters were monitored over three months, i.e., oligo content, pH, appearance of lyophilized formulation and reconstituted solution. The results showed that it is feasible to develop a lyophilized formulation of oligo in water.

A. Phosphate Buffer Preparation (pH 7)

A 0.1 M solution of monobasic sodium phosphate was prepared by dissolving 1.3923 grams of monohydrate material in 100 ml of sterile water. A 0.1 M solution of dibasic sodium phosphate was prepared by dissolving 1.4202 g material in 100 ml of sterile water. 30 ml 0.1 M monobasic sodium phosphate solution and 61 ml 0.1 M dibasic sodium phosphate solution were combined and diluted to 200 ml using sterile water. The pH of the solution was measured as 7.08 at 19.2° C.

B. Sample Preparation & Filtration

862.5 mg of the oligo were dissolved in 55.0245 g the water for injection in a 250 ml beaker by swirling. The solution appeared colorless with a slight haze. The solution was stored at room temperature until filtration, which occurred on the same day. A 50μ aliquot of sample was diluted to 50 ml with the water for injection and the absorbance of this solution was measured at 260 nm on a Hewlett Packard model G1103 UV spectrophotometer. The 0.34361 absorbance units correspond to 11.46 pure oligo mg/ml (73.1% purity). In a separate 250 ml beaker, 862.3 mg oligo were dissolved in 55.0163 g Phosphate buffer, pH7, prepared above by swirling. This solution also appeared colorless with a slight haze and was stored at room temperature until filtration, which occurred on the same day. The absorbance of a 50 μl aliquot of this sample diluted to 50 ml with Phosphate buffer, pH7, was also measured at 260 nm. The absorbance of 0.34688 corresponds to 11.56 pure oligo mg/ml (73.8% purity). Each solution was filtered through a 0.22 μm GV Durapore 250 ml Stericup. The filtered 40 solutions were stored at room temperature prior to filling and lyophilization. 50 μl aliquots of each solution were diluted to 50 ml in their respective diluting solvents (water for injection or Phosphate buffer, pH7). The absorbance of the filtered solutions was measured at 260 nm. Based on the thus obtained absorbances (0.34806 and 0.34518, respectively) the concentrations of pure oligo for filtered samples prepared in the water for injection and pH 7 phosphate buffer were calculated as 11.60 mg/ml (74.0% purity) and 11.51 mg/ml (73.4% purity), respectively.

C. Packaging, Lyophilization & Storage

Based on the results above, 1 ml of each formulation was required in each vial to achieve a concentration of about 12 mg of pure oligo per vial. Using an Eppendorf repeating pipet, 1 ml filtered formulation in the water for injection was delivered to each of 46 Type I 10-ml clear glass vials. For the formulation prepared in Phosphate buffer, pH7, 1 ml aliquots were delivered to each of 45 vials. West 44 16/50 LYO stoppers were placed halfway onto the vials to partially close them. The vials were lyophilized in a Virtis Genesis SQ25 Super XL freeze drier using the following cycle; freeze temperature: −40° C., additional freeze: 10.0 min, condenser set point: −60.0° C., and vacuum set point 400.0 mTorr.

TABLE 1
Cycle of Lyophilization

| | | | Time | |
|---|---|---|---|---|
| Cycle | Temperature (° C.) | Vacuum (mTorr) | (min) | Status |
| Step #1 | −40.0 | 200.0 | 30.0 | Hold |
| Step #2 | 0.0 | 100.0 | 30.0 | Ramp |
| Step #3 | 0.0 | 100.0 | 420.0 | Hold |
| Step #4 | 20.0 | 75.0 | 20.0 | Ramp |
| Step #5 | 20.0 | 75.0 | 120.0 | Hold |
| Post Heat | 20.0 | 75.0 | 240.0 | |

At the completion of the lyophilization process, the stoppers were fully crimped onto the vials. Four vials of each lyophilized formulation were reserved for initial (t=0) testing. One vial of each formulation was discarded, as they were the lyophilization probe vials. The remaining vials were stored at 40±2° C. (13 vials prepared in each water for injection pH 7 phosphate buffer).

D. Sampling & Analysis

3 vials of each formulation were removed from the chamber at 1 month, 2 months and 3 months intervals. Two of the vials were used for assaying at that interval point, and the third vial was used for appearance testing before and after reconstitution, and for pH testing of the reconstituted solution. After removal of aliquots of the reconstituted assay samples for analysis at each time interval, the vials were re-crimped, one of the two vials was inverted and the other remained upright. After 24 hours in these positions, the assay samples were re-analyzed. Because at the one month time interval, the pipet used to dilute the reconstituted samples was not functioning properly, two contingency samples of each formulation were removed from each chamber at 41 days. Appearance, pH and assay testing was performed on both samples, and the results from these samples are reported for the one month time point.

(2) Results

The results obtained for the stability study are summarized in Tables 2 and 3 below, and representative chromatograms are presented in FIGS. 1 through 4 accompanying this patent.

A. Oligo Formulation in Water for Injection

The storage interval and condition had no impact on the appearance of the lyophilized or reconstituted samples. All the lyophilized samples were cracked, white, disk shaped cakes that had adhered to the vial walls in some places. All the reconstituted solutions appeared colorless with lint. The pH of the reconstituted samples was seen to increase upon storage. The highest pH values were recorded at the 2 month interval, with increases of 0.6 pH units observed for samples stored at 40° C. At the 3 month interval, the pH of the reconstituted samples decreased <0.1 pH units from its corresponding measurement at 2 months. Under all storage conditions, the amount of oligo recovered at t=1 was about 90% of the value at t=0 (91.6% at 40° C.). At t=2 months and t=3 months and all storage conditions, recovery remained essentially constant compared to t=1 month. Impurities/degradants made no appreciable impact on the study. Twenty-four hour room temperature storage of the reconstituted solution in either the upright or inverted position had no effect on the assay value. Recoveries of all such samples ranged from 99.5% to 103.8% of their corresponding initial values. Results of the stability study are summarized in Table 2.

B: Oligo Prototype Formulation in pH 7 Phosphate Buffer

TABLE 2
Stability of Oligo at 40° C. (Water for Injection)

| Attribute | T = 0 | T = 1 month | T = 2 months | T = 3 months |
|---|---|---|---|---|
| Lyophilized Plug | White disk cake | White disk cake | White disk cake | White disk cake |
| Appearance | Cracked | Cracked | Cracked | Cracked Vial |
| | Vial Wall Adher. | Vial Wall Adher. | Vial Wall Adher. | Wall Adher. |
| Reconst. Sol. | Colorless w/Lint | Colorless w/Lint* | Colorless w/Lint | Colorless w/Lint |
| Appearance | | | | |
| Reconst. Solution | 7.384 | 7.574*** | 7.986 | 7.940 |
| pH | | | | |
| Assay | 2.277^{a} & 2.066^{b} | 1.954^{a} & 2.025^{b} | 1.988^{a} & 1.993^{b} | 1.970^{a} & 1.964^{b} |
| mg oligo/ml | 2.172 | 1.990 | 1.991 | 1.967 |
| Average | (112.4%)* | (91.6%) | (average 91.7%) | (90.6%) |
| (% initial) | | | | |
| % Total Area | ND | <0.1% | 0.1% | 0.2% |
| Degradants/Impurities | | | | |
| Assay + 24 hr Upright mg | 2.298^{a} | 2.009^{a} | 1.980^{a} | 1.967^{a} |
| oligo/ml | (100.9%) | (102.8%) | (99.6%) | (99.8%) |
| (% t = 0 assay) | | | | |
| % Total Area | ND | <0.1% | 0.1% | 0.2% |
| Degradants/Impurities | | | | |
| Assay + 24 hr Inverted mg | 2.059^{b} | 2.042^{b} | 1.995^{b} | 1.961^{b} |
| oligo/ml | (99.7%) | (100.8%) | (100.1%) | (99.8%) |
| (% t = 0 assay) | | | | |
| % Total Area | ND | 0.1% | 0.1% | 0.3% |
| Degradants/Impurities | | | | |

^{a} and ^{b} represent individual sample vials
“assay + 24 hours”, the sample vial is compared to its respective “assay” vial (i.e., upright vial is compared to assay vial “a” and inverted vial is compared to assay vial “b”)
ND: none detected
*% label claim (1.933 mg oligo/mL) for t = 0
**more stringent of two observations
***average of two readings

The storage interval and condition had a slight impact on the appearance of the lyophilized samples at 40° C. (the cake was cracked and free from the wall). All reconstituted solutions appeared colorless with lint. The pH of reconstituted samples also increased on storage. Again, the highest pH values were recorded at the 2 month interval. At the 3 month interval, the pH of the reconstituted samples also decreased greater or equal to 0.1 pH units from its corresponding measurement at 2 months. Samples stored at 40° C., the amount of oligo recovered compared to t=0 decreased throughout the study from 88.2% at t=1 month to 88.1% at t=2 months and 81.1% at t=3 months. Twenty-four hour room temperature storage of reconstituted solutions in either the upright or inverted position had no effect on the assay value. Recoveries for these samples ranged from 98.9% to 105.4% of their corresponding initial values. The total area percent of degradants/impurities increased with increasing time intervals from 1.7% at t=1 month to 3.5% at t=2 months and 5.9% at t=3 months. Each value was essentially unchanged after 24 hours of upright and inverted room temperature storage of the reconstituted samples. Since all samples were filtered through a 0.22 μm filter and the filtrate was clear, the lint appearing in the reconstituted samples may have come from the vials/stoppers or environmental contamination during reconstitution. Results of the stability study are summarized in Table 3 below.

TABLE 3
Oligo Stability at 40° C. (Phosphate Buffer, pH 7)

| Attribute | T = 0 | T = 1 month | T = 2 months | T = 3 months |
|---|---|---|---|---|
| Lyophilized Plug | White solid cake | White solid cake | White solid cake | White solid cake |
| Appearance | Disk shaped Free | Disk shaped Free | Disk shaped Free | Disk shaped Free |
| | from vial walls | from vial walls | from vial walls | from vial walls |
| Reconst. Sol. Appear. | Colorless Sol. | Colorless Sol. w/ | Colorless Sol. | Colorless Sol. |
| | w/lint | lint** | w/lint | w/lint |
| Reconst. Sol. pH | 7.194 | 7.271*** | 7.347 | 7.236 |
| Assay | 2.048^{a} & 2.070^{b} | 1.818^{a} & 1.815^{b} | 1.828^{a} & 1.810^{b} | 1.690^{a} & 1.648^{b} |
| mg oligo/ml | 2.059 | 1.817 | 1.814 | 1.669 |
| Average | (107.4%)* | (88.2%) | (88.1%) | (81.1%) |
| (% initial) | | | | |
| % Total Area | ND | 1.7% | 3.5% | 5.9% |
| Degradants/Impurities | | | | |
| Assay + 24 hr Upright | 2.057^{a} | 1.802^{a} | 1.8070^{a} | 1.713^{a} |
| mg oligo/ml | (100.4%) | (99.1%) | (98.9%) | (101.4%) |
| (%; t = 0 assay) | | | | |
| % Total Area | ND | 1.6% | 3.6% | 5.4% |
| Degradants/Impurities | | | | |
| Assay + 24 hr Inverted | 2.093^{b} | 1.913^{b} | 1.835^{b} | 1.664^{b} |
| mg oligo/ml | (101.1%) | (105.4%) | (101.4%) | (101.0%) |
| (% t = 0 assay) | | | | |
| % Total Area | ND | 1.3% | 3.3% | 6.5% |
| Degradants/Impurities | | | | |

^{a} and ^{b} represent individual sample vials
“assay + 24 hours”, the sample vial is compared to its respective “assay” vial (i.e., upright vial is compared to assay vial “a” and inverted vial is compared to assay vial “b”)
ND: none detected
*% label claim (1.918 mg oligo/mL) for t = 0
**more stringent of two observations
***average of two readings

(3) Conclusions

The results of this study show that a lyophilized formulation of oligo in water is stable, and that the water formulation is superior to the phosphate buffer formulation in terms of stability at 40° C.

### Example 3

### Spray Drying of Oligo (2)

Excipient-free oligo (SEQ ID NO: 1) solution is prepared by ultrafiltration and diafiltration into a concentration of 50 g/L, and then appropriate amounts of excipients are added to prepare a desired formulation. The oligo solution is filtered with a 0.22-μm filter before use. Excipients used in study include mannitol, trehalose, sucrose, histidine and glycine. They are obtained from Sigma and are used as supplied.

Spray drying is performed using a Model 190 Buchi mini spray dryer (Brinkmann). Using compressed air from an in-house supply, a two-fluid nozzle (0.5 mm) atomized the oligo solution. The air is filtered through a 0.22-μm Milidisk filter (Millipore) before entering the nozzle, and the flow rate is controlled by a variable area flow meter (Cole Parmer, 150 mm). A peristaltic pump (1-100 rpm, Masterflex, Cole Parmer) pumps liquid oligo feed to the nozzle using silicone tubing (3 mm ID). Cooling water is circulated through a jacket around the nozzle. Some modifications are made on the original design for a scale-up operation, which include the replacement of the bag-filter unit with a vacuum cleaning unit (Model 005, VAC-U-MAX, Belleville, N.J.) and relocation of the aspirator to the drying air input. The standard operating condition is: Tᵢₙₗₑₜ (inlet air temperature) of 105° C., Q_{DA} (drying air flow rate) of 1000 L/min, Q_{AA} (atomizing air flow rate) of 1050 L/hr, and Q_{LF} (liquid feed rate) of 15 mL/min. This condition results in a Tₒᵤₜₗₑₜ (outlet air temperature) of 50-55° C.

A two-fluid nozzle (the same nozzle used in spray drying) or an ultrasonic nozzle (Soniteck) is used for atomization. When the two-fluid nozzle is used, warm water (45° C.) is circulated to keep the liquid from freezing in the nozzle. A 3-L two-neck, round-bottom flask is filled with liquid nitrogen and is submerged in a container also containing liquid N₂. The liquid N₂ in the flask is agitated using a magnetic stirrer bar. The nozzle is pointed into the flask in the central neck which is wrapped with a heating tape to avoid nozzle head freezing. The oligo liquid is atomized using an atomizing air flow rate of 1050 L/hr. Sprayed droplets freeze upon contacting liquid N₂. The liquid feed rate is 15 mL/min for air atomization and 5 mL/min for ultrasonic atomization. After spraying, the whole content in the flask is poured into a metal tray and placed in a lyophilizer (GT20) which is pre-chilled to −50° C. After a hold period of one hour at −50° C., vacuum is applied to the chamber. The shelf temperature is increased to −25° C. over a two-hour period and held for 40 hours. During secondary drying, the shelf temperature is increased to 20° C. over a four-hour period and is held for another 20 hours.

### Example 4

### Spray Freeze Drying of Oligo

The oligo liquid product, containing of 10 mg oligo/mL, 100 mM sodium chloride, 50 mM sodium acetate, 0.9% benzyl alcohol, 0.2% polysorbate 20 at pH 5.4, is buffer-exchanged in 10 mM histidine (pH 5.5), and is concentrated to an oligo concentration of 17.7 mg/mL. The second source is from S-Sepharose pool containing 15-25 mg/mL of oligo in 200 mM citrate at pH 6.0. This pool is first buffer-exchanged into 200 mM sodium chloride, 230 mM L-arginine and 10 mM histidine (pH 7.3) to remove citrate. It is then diafiltered into 230 mM L-arginine and 10 mM histidine (pH 7.3) and concentrated to an oligo of concentration of 30 mg/mL. All formulations are prepared using ultrafiltration/diafiltration, followed by the additions of excipients. Varying amounts of carbohydrates (trehalose or mannitol) and amino acids (histidine and/or L-arginine) are used to prepare inhalation formulations as follows:

(a) The oligo liquid product;

(b) oligo, 10 mM histidine, pH 5.5;

(c) oligo, 10 mM histidine, 230 mM L-arginine, pH 7.3;

(d) oligo:trehalose at 60:40 (weight ratio), 10 mM histidine, pH 5.5;

(e) oligo:trehalose at 60:40 (weight ratio), 10 mM histidine and 230 mM L-arginine pH7.4.

Ultrafiltration and diafiltration is performed on a bench-top tangential flow-filtration system (stainless steel Pellicon-2™, Millipore) with a 5 kD regenerated cellulose membrane cassettes with a membrane area of 0.1 m². Eight diavolumes are used in the diafiltration step. Diavolume is defined as the passage of a quantity of buffer equivalent to the volume of retentate (Chang et al. (1996) supra). The experiments are conducted at constant retentate pressure of 18 psi, feed flow rate of 0.5 L/min and at ambient temperature. Some ultrafiltration and diafiltration runs are performed in a fully automated tangential flow filtration system. Details of the system is described elsewhere (Townsend et al. (1988) supra).

The spray freeze-dried oligo powders are prepared using a two-fluid nozzle (from a Buchi 190 spray dryer) for atomization. A 3-L two-neck, round-bottom flask is filled with liquid nitrogen and is submerged in a container also containing liquid N₂. The liquid N₂ in the flask is agitated using a magnetic stirrer bar. The nozzle is pointed into the flask in the central neck which is wrapped with a heating tape to avoid nozzle head freezing. The oligo liquid is atomized using an atomizing airflow rate of 1050 L/hr. Sprayed droplets freeze upon contacting liquid N₂. The liquid feed rate is 10 mL/min for air atomization. After spraying, the whole content in the flask is poured into a metal tray and placed in a lyophilizer (GT20) which is pre-chilled to −50° C. After a hold period of one hour at −50° C., vacuum is applied to the chamber. The shelf temperature is increased to −25° C. over a two-hour period and held for 40 hours. During secondary drying, the shelf temperature is increased to 20° C. over a four-hour period and is held for another 20 hours.

The spray freeze-dried powder is blended with 100 M lactose coarse carrier at 1:10 w/w active oligo:coarse carrier by mixing (Turbula, Glenn Mill) and sieving (250-μm mesh). Ten individuals of pre-weighted samples of 10 mg blended powder (or 5 mg raw powder) are loaded into a dry powder inhaler (Dura Pharmaceuticals, San Diego) and dispersed into a multi-stage liquid impinger (MSLI) at an air flow rate of 60 L/min and an inhalation time of 5 seconds, as outlined above. The MSLI throat piece is attached to the top of the first stage. A filter paper is placed underneath stage 4 to capture fine particles in range of less than 1 μm. The material which deposited in the throat piece and the filter and their washings analyze for oligo content. The fine particle fraction is defined as powder with an aerodynamic mass median diameter of less than 6.8 μm, and is determined by the percentage of oligo which deposited on stages 3, 4 and the filter.

Samples are stored in open glass vials inside sealed desiccators which contain saturated salt solution to control the humidity: calcium chloride at 38% relative humidity (rh). Temperatures are maintained by placing the sealed containers in constant, controlled temperature storage cabinets. Samples of both raw powders and formulated blends are stored at 2-8° C. and at 30° C. The powders are assayed for soluble aggregates, oxidation, and aerosol performance at t=0, 4 weeks, and 24 weeks of storage. The effect of spray drying on oligo aggregation and oxidation is investigated by size exclusion and reverse phase HPLC.

The spray-freeze dried powder is blended with 100 M lactose carriers prior to fine particle fraction (<6.8 μm) measurement using a multi-stage liquid impinger model. Blending can theoretically improve the fine powder's flow properties. Small particles tend to interact with themselves (agglomeration) and with any contact surfaces due to high surface energy. Agglomerated particles behave like large particles and are difficult to be dispersed. Sticking to other contact surfaces results in material loss and poor powder flowability. If the interaction between the spray-dried particle (raw powder) and the carrier particle (F_{r-c}) overcomes the interaction among the raw powder (Fᵣᵣ), it can result in homogeneous blending, thereby enhancing the powder's flowability.

Five formulations ((a), (b), (c), (d) and (e)) are tested for preparing spray freeze-dried oligo powders. Spray freeze drying produces large, porous particles with significantly improved aerosol performance compared to spray-dried powders. The size of these powders is indeed larger than their spray-dried counterpart. The formulations (b) and (d) are selected for further investigation because the arginine-containing powders collapse upon storage in the vials and oligo aggregated (around 6%) in the formulation (a). The FPF of the powders (Formulations (b) and (d)) almost triple compared to their spray-dried counterpart. After storage at 2-8 and 30° C. up to 6 months, the aerosol performance of the blended powders either remains unchanged or becomes slightly better. Another interesting finding is that the nonblended (raw) powder has a better dispersibility than the blended powder, which is opposite to the spray-dried powder. This suggests that spray freeze drying produces aerosol powders of very different aerodynamic properties.

### Example 5

### Spray Freeze Drying of Oligo

Solution is Oligo 50 g/L in H₂O filtered prior to use. A two-fluid nozzle (the same nozzle used in spray drying) or an ultrasonic nozzle (Soniteck) is used for atomization to spray the protein solution into a 3-L two-neck, round-bottom flask full of liquid nitrogen. The whole flask is submerged in liquid N₂ to ensure the system's low temperature. The liquid N₂ in the flask is agitated using a magnetic stirrer bar. Sprayed droplets froze upon contacting liquid N₂. The protein liquid is atomized using an atomizing air flow rate of 1050 L/hr. The liquid feed rate is 15 mL/min for air atomization and 5 mL/min for ultrasonic atomization. Continuous addition of fresh liquid nitrogen into the flask will alleviate this problem. After spraying, the whole content in the flask is poured into a metal tray and placed in a lyophilizer (GT20) which has been pre-chilled to −50° C. After a hold period of one hour at −50° C., vacuum is applied to the chamber. The shelf temperature is increased to −25° C. over a two-hour period and held for 40 hours. During secondary drying, the shelf temperature is increased to 20° C. over a four-hour period and is held for another 20 hours.

### Example 6

### Powder Formation by SEDS (Solution Enhanced Dispersion by Supercritical Fluids) of Oligo

The underlying principle of the process is based on dispersing an aqueous solution, which contains the biomaterial, with supercritical carbon dioxide and a polar organic solvent in a three-channeled coaxial nozzle. The supercritical CO₂ is used to extract the aqueous phase from the product. Because water is not soluble in pure supercritical CO₂, an additional organic solvent, that is miscible with water as well as supercritical CO₂ is needed. The organic solvent acts both as precipitating agent and as modifier, enabling the non-polar CO₂, to remove the water. The high dispersion in the jet at the nozzle outlet facilitates rapid (<1 s) formation of dry particles of small size.

Oligo (50 g/L) in water with no excipients or a minimal NaCl (0.005M) content contains the plasmid (50 mg/L) and mannitol as inert excipient (50 g/L) for all experiments. Sodium chloride (0.6M) or sodium acetate (0.04M) are added to the aqueous solution for some experiments as discussed below. Carbon dioxide is taken from a cylinder with dip tube in liquid form and cooled to ca. ˜18° C. to maintain liquid state during pumping. It is brought to operating temperature (50° C.) in a heat exchanger before entering the nozzle. The feed rates are 0.03 mL/min, 0.9 mL/min and 10 mL/min for the aqueous solution, organic solvent and liquid CO₂, respectively. The nozzle is mounted on a cylindrical stainless steel particle formation vessel (50 mL). A filter paper (Whatman International Ltd, Maidstone, UK) at the bottom of the vessel retains the particles, while the water is removed together with the carbon dioxide and the organic solvent in a ternary mixture. The back pressure regulator controls the operating pressure in the vessel (200 bar) and expands the mixture leaving the vessel to atmospheric pressure.

### Example 7

### Emitted Dose Studies of Spray-Dried Oligo

Three batches of EPI-2010 were spray dried as previously described. HPLC analysis with UV detection was used to confirm that there was no degradation of the material following particle size reduction.

The emitted dose studies consisted of collecting the EPI-2010 drug powder in Nephele tubes and assaying the collected drug by HPLC. Triplicate experiments were performed at each airflow rate for each of the three dry powder inhalers tested, i.e., Rotahaler, Diskhaler and multi-dose DPI.

To collect the emitted dose of EPI-2010 drug from the respective dry powder inhaler being tested, a Nephele tube was fitted at one end with a glass filter (Gelman Sciences, Type A/E, 25 mm), which, in turn, was connected to the airflow line. At the other end of the Nephele tube was secured a silicone adapter, which has an opening to receive the mouthpiece of the respective dry powder inhaler being tested. The desired airflow, i.e., 30, 60, or 90L/min, was achieved through the Nephele tube. At that point, the respective dry powder inhaler's mouthpiece was inserted into the silicone rubber adapter. Airflow was continued for ˜four seconds. After four seconds of airflow through the Nephele tube, the tube was removed and the tube end-caps were screwed onto the ends of the tube. The end-cap of the tube, not containing the filter, was removed and either 10 ml or 20 ml of HPLC grade water was added to the tube. The tube end-cap was reattached and the tube shaken for ˜1.0 minute.

Following shaking, the end-cap was removed from the tube and the solution was transferred to a 10 ml plastic syringe fitted with a syringe filter (Cameo 13N Syringe Filter, Nylon, 0.22 micron). An aliquot of the solution was directly filtered into an HPLC vial for eventual EPI-2010 assay via high pressure liquid chromatography.

The emitted dose experiments were performed with ˜12.5 mg of the spray-dried EPI-2010 drug being placed in either a gelatin capsule (Rotahaler), a clean Ventodisk blister (Diskhaler) or a clean Aclar blister used in the IDL multi-dose device.

Table 4 shows the average percentage of emitted dose obtained for each of the triplicate experiments conducted with the different devices at the different airflow rates.

It can be seen in Table 4 that the emitted dose values was generally high for all three devices. The emitted dose from the IDL multi-dose device at the different air flow rates tested are in general higher than those obtained with either the Diskhaler or Rotahaler.

TABLE 4
Percent Emitted Dose with Different Devices and Flow Rates

| | Date of Experiment | Flow Rate | Emetted Dose |
|---|---|---|---|
| Device | (Volume, ml) | (L/min) | (%) |
| Rotahaler | Aug. 22, 2001 (20) | 30 | 7.7 |
| | Aug. 16, 2001 (20) | 60 | 21.5 |
| | Aug. 23, 2001 (20) | 90 | 42.8 |
| Diskhaler | Aug. 27, 2001 (20) | 30 | 18.2 |
| | Aug. 24, 2001 (20) | 60 | 34.3 |
| | Jul. 23, 2001 (10) | 90 | 56.5 |
| | Aug. 3, 2001 (10) | 90 | 60.7 |
| | Aug. 8, 2001 (20) | 90 | 63.2* |
| Multi-Dose | Aug. 16, 2001 (20) | 30 | 33.2 |
| | Aug. 15, 2001 (20) | 60 | 72.5 |
| | Jul. 24, 2001 (10) | 90 | 66.8 |

*Only a single emitted dose experiment, thus no average of three experiments

### Example 8

### Long-Term Stability of Excipient-Free Oligo (EPI-2010)

The EPI-2010 anti-sense compound was prepared as an excipient-free lyophilization product in sterile glass vials containing 10 mg/vial. The product was put up on stability according to ICH Guidelines.

Results of the testing are presented in Tables 5-(1) and (2) below. The data indicate that the EPI-2010 anti-sense product is very stable to the lyophilization process (freeze-drying) in the absence of any cryoprotectants or excipients, and it remains stable for at least 18 months in a dry form.

TABLE 5-1
Stability Report (1)
Product Name: EPI-2010 Drug Product, 10 mg/vial
Storage Condition: 5 ± 3° C., stored upright

| | | Stability Interval (Months) and Interval Pull Dates | | | |
|---|---|---|---|---|---|
| | | 1 | 3 | 6 | 9 |
| Test | Time 0² | 17 Jul 00 | 15 Sep 00 | 15 Dec 00 | 15 Mar 01 |
| Appearance | Pass | Pass | Pass | Pass | Pass |
| Lyophilized product | Pass | Pass | Pass | Pass | Pass |
| Reconstituted solution | (02 Jun 00) | (17 Jul 00) | (18 Sep 00) | (20 Dec 00) | (16 Mar 01) |
| Purity by CGE (%) | | | | | |
| Full-length 21-mer^{b} | 92 | 90 | 93 | 93 | 92 |
| | (07 Aug 00) | (31 Jul 00) | (04 Oct 00) | (18 Dec 00) | (16 Mar 01) |
| Purity by HPLC (%) | | | | | |
| Full-length 21-mer^{d} | 96 | 95 | 94 | 95 | 98 |
| Total oligonucleotide | 4 | 5 | 6 | 5 | 2 |
| impurities | (12 Jun 00) | (17 Jul 00) | (18 Sep 00) | (18 Dec 00) | (16 Mar 01) |
| Purity by ³¹P-NMR^{e} (%) | | | | | |
| P = S content | >99.5 | — | — | >99.5 | |
| P = O impurities | <0.5 | — | — | <0.5 | — |
| | (30 Jun 00) | | | (08 Jan 01) | |
| Oligonucleotide content | 101 | 96 | 96 | 96 | 96 |
| by UV absorbance at | (31 May 00) | (17 Jul 00) | (18 Sep 00) | (18 Dec 00) | (16 Mar 01) |
| 260 nm (%) | | | | | |
| pH | 6.3 | 6.2 | 6.2 | 6.3 | 6.2 |
| | (02 Jun 00) | (17 Jul 00) | (18 Sep 00) | (20 Dec 00) | (16 Mar 01) |

TABLE 5-2
Stability Report (2)

| | Stability Interval (Months) and Interval Pull Dates | | | |
|---|---|---|---|---|
| | 12 | 18 | 24 | 36 |
| Test | 15 Jun. 2001 | 17 Dec. 2001 | | |

| | | |
|---|---|---|
| Appearance | | |
| Lyophilized product | Pass | Pass |
| Reconstituted solution | Pass | Pass |
| | (19 Jun. 2001) | (18 Dec. 2001) |
| Purity by CGE (%) | | |
| Full-length 21-mer^{d} | 93 | 90 |
| | (18 Jun. 2001) | (18 Dec. 2001) |
| Purity by HPLC (%) | | |
| Full-length 21-mer^{d} | 98 | 98 |
| Total oligonucleotide | 2 | 2 |
| impurities | (18 Jun. 2001) | (18 Dec. 2001) |
| Purity by ³¹P-NMR^{e} | | |
| P═S content | — | — |
| P═O impurities | — | — |
| Oligonucleotide | 96 | 96, 96, 98^{g} |
| content by UV | (18 Jun. 2001) | (21 Dec. 2001) |
| absorbance at 260 nm | | (03 Jan. 2002) |
| (%) | | |
| pH | 6.3 | 6.2 |
| | (19 Jun. 2001) | (18 Dec. 2001) |

### Example 9

### Lyophilization of Oligo Followed by Milling

The oligonucleotide is dissolved to a concentration of 20 mg/mL in sterile water for injection. It is passed through a 0.22 μM filter. 500 mL of solution are dispensed into 2000 mL Virtis lyophilizer jars, and shell frozen in a bath of dry ice and methyl alcohol. The lyophilizer jars are then attached to a Virtis lyophilizer with a vacuum gauge reading of <100×10⁻³ microns. Lyophilization is considered complete when no cold spots remain on the jars, or approximately 5 days. The dried oligonucleotide (10 gm per jar) is transferred to Nalge 4L wide-mouth bottle and stored frozen until particle size reduction is attempted.

Following lyophilization, the course particles of oligonucleotide are placed into a vibratory feeder and fed into a Model 00 Jet-O-Mizer mill (Fluid Energy Aljet, Telford, Pa.) operated under 100 to 120 psi of nitrogen. Fine particles are collected in a filter bag.

Fine particles are further adjusted to a narrower range by high velocity air stream and centrifugal force by feeding the milled oligonucleotide into a Roto-Sizer (Fluid Energy Aljet, Telford, Pa.).

### Example 10

### Design and Synthesis of Oligo

The anti-sense oligonucleotide I (oligo I) or EPI-2010 having SEQ ID NO: 1 referred to in this specification is targeted to the human A₁ adenosine receptor mRNA. Anti-sense oligo I is 21 nucleotide long, overlaps the initiation codon, and has the following sequence: 5′-GAT GGA GGG CGG CAT GGC GGG-3′ (SEQ ID NO: 1). The oligo I was previously shown to abrogate the adenosine-induced bronchoconstriction in allergic rabbits, and to reduce allergen-induced airway obstruction and bronchial hyperresponsiveness (BHR), as discussed above and shown by Nyce, J. W. & Metzger, W. J., Nature, 385:721 (1977), the relevant portions of which reference are incorporated in their entireties herein by reference (See WO 00/09,525). The oligo I was synthesized to have phosphorothioate backbones using an Applied Biosystems 394 synthesizer (Perkin Elmer, Calif.). The oligo I may be purified using NENSORB chromatography (DuPont, Md.).

These are clearly superior results, which could not have been expected based on the knowledge of the art at the time of this invention. The experimental data and results provided are clearly enabling of the effect of ubiquinones on adenosine cellular levels and, therefore, on its therapeutic affect on diseases and conditions associated with them, as described and claimed in this patent.

The foregoing examples are illustrative of the present invention, and are not to be construed as limiting thereof. The invention is defined by the following claims, with equivalents of the claims to be included therein.

What is being claimed as novel & unobvious in Letters Patent of the United States is:

1. A dry powder formulation, comprising an agent comprising oligonucleotide(s) (oligo(s)) and having greater than 80% particles of about 0.1μ to about 100μ in diameter.

2. The formulation of claim 1, wherein greater than 85% particles are about 0.1μ to about 100μ in diameter.

3. The formulation of claim 1, wherein greater than 90% particles are about 0.1μ to about 100μ in diameter.

4. The formulation of claim 1, wherein greater than 80% particles are about 0.1μ to about 10μ in diameter.

5. The formulation of claim 1, wherein greater than 85% particles are about 0.1μ to about 10μ in diameter.

6. The formulation of claim 1, wherein greater than 90% particles are about 0.1μ to about 10μ in diameter.

7. The formulation of claim 1, wherein greater than 80% particles are about 0.1μ to about 5μ in diameter.

8. The formulation of claim 1, wherein greater than 85% particles are about 0.1μ to about 5μ in diameter.

9. The formulation of claim 1, wherein greater than 90% particles are about 0.1μ to about 5μ in diameter.

10. The formulation of claim 1, wherein greater than 80% particles are about 10μ to about 50μ in diameter.

11. The formulation of claim 1, wherein greater than 85% particles are about 10μ to about 50μ in diameter.

12. The formulation of claim 1, wherein greater than 90% particles are about 10μ to about 50μ in diameter.

13. The formulation of claim 1, wherein greater than 80% particles are about 10μ to about 40μ in diameter.

14. The formulation of claim 1, wherein greater than 85% particles are about 10μ to about 40μ in diameter.

15. The formulation of claim 1, wherein greater than 90% particles are about 10μ to about 40μ in diameter.

16. The formulation of claim 1, wherein the oligo(s) is (are) about 4 to about 200 mononucleotide long.

17. The formulation of claim 1, wherein the oligo(s) is (are) about 8 to about 30 mononucleotide long.

18. The formulation of claim 1, wherein the oligo(s) comprise(s) sense oligo(s).

19. The formulation of claim 1, wherein the oligo(s)comprises anti-sense oligo(s).

20. The formulation of claim 1, wherein the oligo(s) comprises deoxynucliec acids.

21. The formulation of claim 1, wherein the oligo(s) comprise(s) ribonucleic acids.

22. The formulation of claim 1, wherein the oligo(s) comprise(s) a single stranded oligo(s).

23. The formulation of claim 1, wherein the oligo(s) comprise(s) a double stranded oligo(s).

24. The formulation of claim 19, wherein the anti-sense oligo(s) hybridize(s) to a polynucleotide target comprising genes, genes' initiation codons, genomic flanking regions, intron-exon borders, their 5′-end, their 3′-end, or regions within 2 to 10 nucleotides of the 5′-end or 3′-end, the juxta-section between coding and non-coding regions, or coding and non-coding regions of RNAs corresponding to the target genes.

25. The formulation of claim 19, wherein the anti-sense oligo(s) comprises a multi-targeted oligo that hybridizes to at least two nucleic acid targets.

26. The formulation of claim 18, wherein at least one mononucleotide is substituted or modified by one or more of phosphorothioate, chiral phosphorothioate, phosphorodithioate, phosphotriester, aminoalkylphosphotriester, methyl phosphonate, 3′-alkylene phosphonate, chiral phosphonate, phosphinate, phosphoramidate, 3′-amino phosphoramidate, aminoalkylphosphoramidate, thionophosphoramidate, thionoalkylphosphonate, thionoalkylphosphotriester, boranophosphate, morpholino, siloxane, sulfide, sulfoxide, sulfone, formacetyl, thioformacetyl, methylene formacetyl, thioformacetyl, alkene, sulfamate, methyleneimino, methylenehydrazino, sulfonate, sulfonamide, amide, thioether, carbonate, carbamate, sulfate, sulfite, hydroxylamine, methylene(methylimino), methyleneoxy (methylimino), 2′-O-methyl, or phosphoramidate residues, or combinations thereof.

27. The formulation of claim 26, wherein all mononucleotides are substituted or modified.

28. The formulation of claim 19, wherein the anti-sense oligo comprises SEQ ID NO: 1.

29. The formulation of claim 28, wherein the cell-internalized or up-taken agent comprises transferring, asialoglycoprotein or streptavidin.

30. The formulation of claim 29, wherein the oligo(s) is operatively linked to a cell-internalized or up-taken agent or to a eukaryotic or prokaryotic vector.

31. The formulation of claim 1, consisting essentially of the agent(s).

32. The method of claim 1, wherein the formulation further comprises an agent selected from carriers or diluents, bulking agents, preservatives, stabilizers, flowability improving agents, cohesiveness improving agents, surfactants, other bioactive agents, coloring agents, aromatic agents, flavoring agents, anti-oxidants, fillers, volatile oils, dispersants, buffering agents, RNA inactivating agents, propellants or preservatives.

33. A method for delivering a dry powder formulation to a target tissue or organ, comprising systemically or topically administering to a subject an effective amount of the dry powder formulation of claim 1.

34. The method of claim 33, wherein the formulation is administered into the subject's respiratory system.

35. The method of claim 33, wherein the formulation is administered by inhalation.

36. The method of claim 33, wherein the formulation is administered nasally.

37. The method of claim 33, wherein the formulation is instilled into the subject's lungs.

38. The method for treating a subject afflicted with a disease or condition comprising administering to the subject the dry powder formulation obtainable in claim 1, wherein the agent is administered in a prophylactic or therapeutic amount.

39. The method of claim 38, wherein the disease or condition are associated with bronchoconstriction, allergy, cancer and/or inflammation of the lung.

40. The method of claim 33, wherein the formulation is administered to a subject together with one or more at least on other therapeutic agent(s).

41. The method of claim 40, wherein the therapeutic agent(s) comprise(s) adenosine A₁, A_{2b} and A₃ receptor inhibiting agents and adenosine A₂ₐ receptor stimulating (agonist) agents, anti-inflammatory agents, anti-bacterial agents, anti-sepsis agents, anti-allergic rhinitis agents, kidney activity maintenance and restoration agents and agents for the treatment of pulmonary vasoconstriction, inflammation, allergies, asthma, impeded respiration, respiratory distress syndrome (RDS and ARDS), pain, cystic fibrosis, pulmonary hypertension, pulmonary vasoconstriction, emphysema, chronic obstructive pulmonary disease (COPD), and cancers selected from the group consisting of leukemias, lymphomas and carcinomas of the colon, breast, lung, pancreas, hepatocellular carcinoma, kidney, melanoma, liver, lung, breast and prostate metastatic cancer, radiation agents, chemotherapeutic agents, imaging agents, cardiac stress testing agents, antibody therapy agents, phototherapeutic agents, adenosine, and other anti-arrhythmic agents.

42. The method of claim 38, wherein the formulation is administered orally, intracavitarily, intranasally, intraanally, intravaginally, intrauterally, intraarticularly, transdermally, intrabucally, intravenously, subcutaneously, intramuscularly, intravascularly, intratumorously, intraglandularly, intraocularly, intracranial, into an organ, intravascularly, intrathecally, intralymphatically, intraootically, by implantation, by inhalation, intradermally, intrapulmonarily, intraoptically, by slow release, by sustained release and by a pump.

43. The method of claim 38 wherein the subject is a mammal.

44. The method of claim 43, wherein the mammal is a human or non-human animal.

45. The method of claim 38, wherein the dry powder formulation is administered in amount of about 0.005 to about 150 mg/kg body weight.

46. The method of claim 45, wherein the dry powder formulation is administered in amount of about 0.01 to about 75 mg/kg body weight.

47. The method of claim 46, wherein the dry powder formulation is administered in amount of about 1 to about 50 mg/kg body weight.

48. The method of claim 38, which comprises a prophylactic or therapeutic method.

49. The method of claim 38, wherein the disease or condition comprises sepsis, pulmonary vasoconstriction, inflammation, allergies, asthma, impeded respiration, respiratory distress syndrome, Acute Respiratory Distress Syndrome (ARDS), renal damage or failure associated with ischemia or the administration of drugs or radioactive agents, side effects of adenosine or other anti-arrhythmic agents administered to treat arrhythmias or SupraVentricular Tachycardia (SVT), or to test cardiovascular function, ischemia, pain, cystic fibrosis (CF), pulmonary hypertension, pulmonary vasoconstriction, emphysema, chronic obstructive pulmonary disease (COPD), allergic rhinitis (AR) and cancers selected from the group consisting of leukemias, lymphomas and carcinomas of the colon, breast, lung, pancreas, hepatocellular carcinoma, kidney, melanoma, hepatic, lung, breast and prostate, metastatic cancer, or those that are treated with radiation, chemotherapeutic, antibody therapy or phototherapeutic agents.

50. A method of preparation of a dry powder formulation of an agent(s) comprising an oligonucleotide (oligo), comprising
obtaining a dry pharmaceutical agent(s) comprising an oligonucleotide(s) (oligo(s));
altering the particle size of the agent(s) to about 0.01 to about 1000μ in diameter and an average particle size about 0.1μ to about 100μ in diameter; and
selecting particles of the agent greater than about 80% about 0.1μ to about 100μ in diameter.

51. The method of claim 50, wherein the obtained agent is(are) in solid form.

52. The method of claim 51, wherein the solid agent(s) comprise(s) a powder.

53. The method of claim 50, wherein the particle size is altered by milling.

54. The method of claim 50, wherein the particle size is altered by jet milling.

55. The method of claim 50, wherein the particle size is altered by fluid energy milling.

56. The method of claim 50, wherein the particle size is altered by sieving.

57. The method of claim 50, wherein the particle size is altered by homogenization.

58. The method of claim 50, wherein the particle size is altered by granulation.

59. The method of claim 50, wherein the particle size is altered by milling, homogenization or granulation, the method further comprising sieving the formulation.

60. The method of claim 50, further comprising storing the thus obtained formulation under controlled conditions of temperature, humidity, light, pressure or other conditions that do not significantly alter the flowability of the agent.

61. The method of claim 50, consisting essentially of the agent(s).

62. The method of claim 50, further comprising placing the agent(s) in solution, suspension or emulsion in a suitable carrier or diluent.

63. The method of claim 50, wherein the agent(s) is(are) placed in solution, suspension or emulsion prior to altering its particle size of the agent(s).

64. The method of claim 50, wherein the particle size is altered after the agent(s) is(are) placed in solution, suspension or emulsion.

65. The method of claim 50, wherein the particle size is altered and selected in a single step.

66. The method of claim 50, wherein the particle size is altered and selected by spray-drying under conditions effective to attain the desired particle size.

67. The method of claim 66, wherein the spray-drying comprises a gas driven jet or employing a jet nebulizer.

68. The method of claim 67, wherein the gas comprises air.

69. The method of claim 50, wherein the particle size is altered by crystallization, precipitation or sonication from the solution, suspension or emulsion.

70. The method of claim 50, wherein the particle size selected by sieving.

71. The method of claim 50, wherein the particle size is selected by lyophilization of the solution, suspension or emulsion.

72. The method of claim 71, wherein the lyophilization comprises spray-lyophilization.

73. The method of claim 50, wherein the particle size is altered and selected by freeze drying under conditions effective to attain the desired particle size.

74. The method of claim 50, wherein the particle size is altered and selected with the aid of a supercritical fluid.

75. A dry powder formulation obtained by the method of claim 50.

76. A delivery device comprising the formulation of claim 1.

77. The device of claim 76, being a dry particle inhalation.

78. The device of claim 76, wherein the device comprises a dry powder inhalation that delivers particle sizes about 0.1μ to about 10μ.

79. The device of claim 76, wherein the device comprises a dry powder inhalation that delivers particle sizes about 10μ to about 100μ.

80. The device of claim 76, wherein the device is a dry powder inhalator adapted for receiving and piercing or opening a capsule(s) or cartridge(s) and producing the dry powder formulation that is provided separately in a piercable or openable capsule(s), or cartridge(s).

81. The device of claim 76, suitable for nasal, inhalable, respirable, intrapulmonary, intracavity or intraorgan delivery.

82. A diagnostic or delivery kit comprising, in separate containers,
a delivery device;
a dry powered formulation of claim 1; and
instructions for delivery of the formulation; and
optionally a therapeutic or diagnostic agent(s) other than the agent(s) comprising the oligo(s), anti-oxidants, fillers, volatile oils, dispersants, anti-oxidants, propellants, preservatives, solvents, buffering agents, RNA inactivating agents, agents that are internalized or up-taken by a cell, flavoring agents, aromatic agent(s), or coloring agents.

83. The kit of claim 82, comprising the delivery device, a surfactant, the dry powder formulation and an other therapeutic agent(s).

84. The kit of claim 82, further comprising in a separate container, a propellant and pressurized means for delivery adapted for delivering the dry powder formulation, and instructions for loading into the delivery device the dry powder formulation and the propellant and pressurized means.


SEQUENCE LISTING (1 sequence)

SEQ ID NO: 1 - DNA, 21 bp, Homo sapiens
gatggagggcggcatggcggg